(19) United States
(12) Patent Application Publication
(10) Pub. No.: US 2021/0286604 A1
(43) Pub. Date: Sep. 16, 2021

(54) METHODS, SERVICES, SYSTEMS, AND ARCHITECTURES TO OPTIMIZE LABORATORY PROCESSES

(71) Applicant: GenoFab, Inc., Fort Collins, CO (US)

(72) Inventor: Jean Peccoud, Fort Collins, CO (US)

(21) Appl. No.: 17/203,690

(22) Filed: Mar. 16, 2021

Related U.S. Application Data
(60) Provisional application No. 62/990,428, filed on Mar. 16, 2020.

(51) Int. Cl. G06F 8/54; G06F 8/30

(52) U.S. Cl. CPC G06F 8/54; G06N 20/00; G06F 8/315

(57) ABSTRACT

The invention described herein is for generating executable program code manifesting a dataflow description in accordance with a set of nodes and links of a flow graph. More specifically, the invention is directed at generating, based on aggregating at least a subset of the plurality of task data objects that may be received, a dataflow description. The generated data flow description having at least one shared attribute. An executable program code may be generated to produce an output data object based on executing, by the processor, the executable program code manifesting the dataflow description.

### CROSS-REFERENCE TO RELATED APPLICATIONS

This application claims the benefit of priority to U.S. Provisional Patent Application No. 62/990,428 filed Mar. 16, 2020, titled “METHOD AND SYSTEMS OF LABORATORY DATAFLOW PROCESSES.” Said U.S. Provisional Patent Application No. 62/990,428 is incorporated by reference in the entirety herein.

### FIELD OF THE INVENTION

Embodiments relate generally to a method and system for generating electronic dataflows. More specifically, disclosed are methods and apparatuses for dataflow engines that optimize laboratory processes.

### BACKGROUND

Web service providers, and provider networks, in general, often allow customers to specify a dataflow that accomplishes a set of computational tasks to solve a given computational problem, logistical problem, or generally any process that may be directed by a computer system. In some cases, a dataflow may also be executed on a local client computing device. Conventional approaches for providing dataflow services usually rely on tools providing for selections of sequences of tasks. However, such conventional approaches fail to account for the complexities of some laboratory processes. For example, throughout the lifecycle of a project, a project team may develop a large amount of project information, including project requirements, assumptions, contacts, and build sheets. A single project may last for months or even years, which makes the tasks of maintaining, organizing, and exporting project information difficult. There also arises a need to reconfigure the process in real time as the project proceeds, based on changed or changing project circumstances. Further, gaining approvals for proposed application and component configurations and tracking these approvals for auditing purposes requires significant expenditures of time and resources. Other difficulties encountered can include a high rate of systematic failure that require the development of contingency plans and fallback strategies in the process development. Yet another challenge is the difficulty of predicting a process performance likelihood of success that requires many iterations of process modeling and experimental validation. Accordingly, there is a need in the art for improved laboratory processes and methods for creating the same.

### SUMMARY

Systems and methods in accordance with the embodiments described herein overcome various deficiencies in existing approaches to electronic dataflows. In particular, various embodiments provide a principled approach to process development in a laboratory setting. For example, in an embodiment of the invention, a start point, end point, and various rules may be used to automatically define a process and generating a dataflow for managing a lab project. For example, a triggering event associated with laboratory project attributes is detected. The laboratory project attributes are evaluated with a trained model to select a dataflow process. A body of project information is generated based on the dataflow process, where the project information can be used to test a scientific hypothesis. Thereafter, the project information can be stored in a unified data model and/or utilized for another purpose.

Accordingly, embodiments provide for a hierarchical approach to process development, making it possible to develop processes using methods proven in other engineering fields that extensively rely on libraries of reusable components corresponding to different abstraction levels. Developing abstraction hierarchies is known to reduce the cost of developing complex systems.

Further, embodiments described herein make it possible to generate a non-ambiguous description of a process. This can be used to share the process internally to teams during the process development phase. This can also be used to provide a non-ambiguous description of the process to a third party. For example, a process development team needs to share the process description with a manufacturing facility. A scientist may want to share a process with collaborators involved in reproducibility studies. It facilitates the requalification of the process by changing individual steps that are well identified.

Further still, while there may be some uncertainty with respect to the biological performance of a process, other parameters like availability of resources, costs, and delays are well known. Approaches described herein make it possible to compare these cost metrics on functionally equivalent processes. This analysis can be performed ahead of launching a process or at runtime, just like navigation applications can determine the optimal itinerary prior to starting a trip and reroute a driver based on evolving traffic conditions while on the way.

Further still, embodiments herein make it possible to automatically generate valid processes to sample the process design space, compare performance, and possibly apply machine learning algorithms to process optimization. Formalizing processes using embodiments described herein increases the reproducibility of research dataflows. Formalized workflows increase reproducibility and reduce experimental errors. This makes it easier to compare alternative processes during process development.

Further still, certain embodiments can reduce the cost of data by providing a framework suitable to divide labor between specialized services. It also reduces the cost of data by reducing the rate of random failure and minimizing the cost of failed experiments by detecting failure early. It can also reduce the cost of project failure by systematically including rework strategies to mitigate the negative effects of systematic failures.

Further still, embodiments described herein can structure data by capturing relations between different data generated by various services. The data underlying structure facilitates their statistical analysis and makes structured datasets more valuable than unstructured data.

Further still, embodiments herein can accelerate the execution of research projects by accelerating the comparison of candidate processes by automatically aggregating and comparing resources shared by different processes.

Further still, various embodiments provide a log of process execution. The log in certain embodiments is more comprehensive than the documentation captured in electronic laboratory notebooks. This can contribute to increasing traceability of the process and can help with documentation supporting patent applications, publications, or regulatory approvals.

Advantageously, dataflow and training systems described herein may allow the system to conserve memory and bandwidth over other systems. For example, utilizing a unified data model to aggregate laboratory project information in a centralized location may allow the system to conserve memory and bandwidth over a system in which pieces of project information are stored in different locations and different formats across the enterprise and must be located and retrieved from these various locations during an export operation. In other embodiments, receiving different types of laboratory project information through specialized interface modules may save processing power and memory over a system that uses generic interface modules to receive project information. These savings may occur because the system may know, without performing an analysis of the received project information, how to format and store the received information.

Various other functions and advantages are described and suggested below as may be provided in accordance with the various embodiments.

### BRIEF DESCRIPTION OF THE DRAWINGS

Various embodiments and illustrative examples are disclosed in the following detailed description and the accompanying drawings:

FIG. 1 illustrates an example environment in which aspects of the various embodiments can be implemented.

FIG. 2 illustrates an example system according to various embodiments.

FIG. 3 illustrates an example data model in accordance with various embodiments.

FIGS. 4A and 4B illustrate a data model to track a sample in accordance with various embodiments.

FIG. 5 illustrates an example of programmatic access to inputs and outputs in accordance with an embodiment.

FIG. 6 is an example process that can be utilized in accordance with various embodiments.

FIG. 7 illustrates an example configuration of components of a device that can be utilized in accordance with various embodiments.

FIG. 8 illustrates, in an example embodiment, a hierarchical organization of data in a catalog structure.

FIG. 9 illustrates, in an example embodiment, task data collected at various steps of a task completion process.

FIG. 10 illustrates, in an example, an assemble fragment step in a gene synthesis dataflow process embodiment.

FIG. 11 illustrates, in an example embodiment, a dataflow process hierarchy in clone matching of a design sequence.

FIG. 12 illustrates, in an example embodiment, a gene synthesis laboratory dataflow process.

FIG. 13 illustrates, in example embodiments, dataflow based strategies for producing a gene variant.

FIG. 14 illustrates an example embodiment of a dataflow process.

### DETAILED DESCRIPTION

Various embodiments or examples may be implemented in numerous ways, including as a system, a process, an apparatus, a user interface, or a series of program instructions on a computer-readable medium such as a computer-readable storage medium or a computer network where the program instructions are sent over optical, electronic, or wireless communication links. In general, operations of disclosed processes may be performed in an arbitrary order, unless otherwise provided in the claims.

A detailed description of one or more examples is provided below, along with accompanying figures. The detailed description is provided in connection with such examples but is not limited to any particular example. The scope is limited only by the claims, and numerous alternatives, modifications, and equivalents are encompassed. Numerous specific details are set forth in the following description in order to provide a thorough understanding. These details are provided for the purpose of example and the described techniques may be practiced according to the claims without some or all of these specific details. For clarity, technical material that is known in the technical fields related to the examples has not been described in detail to avoid unnecessarily obscuring the description.

Embodiments herein include a cloud-based laboratory information management platform based at least on a catalog of objects corresponding to the different types of information used in relation to laboratory operations. The catalog types are classes of objects that share the same data model. Catalog entries are instances of catalog types that are defined by setting the values of configuration variables. In addition, the catalog allows users to track catalog items by recording runtime data associated with the acquisition of catalog entries. The catalog has a hierarchical tree structure, in an embodiment. Its roots correspond to generic classes of objects. Specialized catalog types can be defined as children of more generic classes. Child catalog types add new data fields to the data model inherited from their parent. In addition to vertical relations between records, the catalog supports links between records across the branches of the tree to express lineage relationships between record types.

Each catalog item is associated with a task that reflects the acquisition process. The task status corresponds to the different stages of the item life cycle. Tasks can be used to associate catalog items and laboratory members involved in their acquisition. A task is a basic unit of work. The analysis of task statistics provides insight into the performance of a laboratory as a whole as well as the performance of individual members. It can suggest actions to improve laboratory productivity. In an embodiment, tasks may enable a pay-per-use business model using tasks as the usage metric.

The dataflow platform herein supports the development of services that require the completion of multiple tasks. A service is a path on the graph defined by the data type compatibility of lineage links between catalog entries. The dataflow platform services can be defined in a hierarchical way by allowing complex services to call simpler services. Service interfaces are defined by catalog entries corresponding to the service inputs and outputs. The output of a service can be connected to the input of another service if they have matching data types. The dataflow platform can automate the development of new services using routing algorithms similar to the way navigation apps suggest itineraries that connect a starting point and a destination. This is achieved by comparing paths on the graph defined by data type compatibility between services.

This service architecture supports two models of collaboration between laboratories using the dataflow platform. By publishing a service interface, an organization can provide other platform users means to outsource part of a complex process to an external service provider. Instead of simply publishing the service interface, an organization can transfer its technology by publishing the service itself to allow other users to execute the service in house.

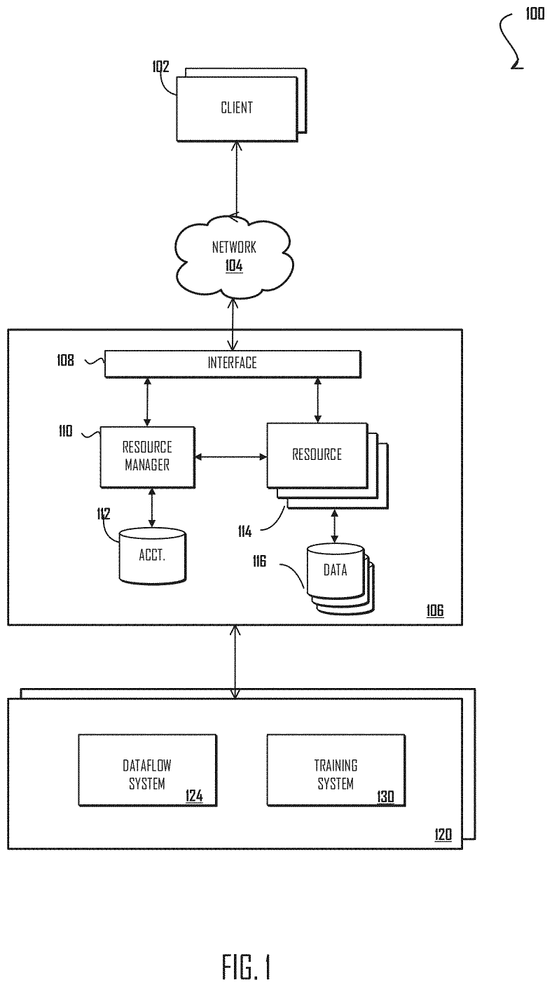

FIG. 1 illustrates an example environment 100 in which aspects of the various embodiments can be implemented. In this example, a user (e.g., project manager or other authorized entity) can utilize a client device 102 to communicate across at least one network 104 with a resource provider environment 106. The client device 102 can include any appropriate electronic device operable to send and receive requests or other such information over an appropriate network and convey information back to a user of the device. Examples of such client devices 102 include personal computers, tablet computers, smartphones, notebook computers, and the like. The user can include a person authorized to manage the aspects of the resource provider environment.

The resource provider environment 106 can provide dataflow management services for managing laboratory operations and the like. This can include, for example, optimizing conventional laboratory management processes and improving the limitations of electronic laboratory notebooks (ELN).

### Laboratory Information Management Systems (LIMS)

In laboratory environments, conventional information management systems (LIMS) have been utilized. In an embodiment, a LIMS can be paper-based and include, for example, a series of log-sheets, physical folders, and other physical record-keeping systems. In various embodiments, a laboratory information management system can be a family of database systems designed to manage data related to laboratory operations. LIMS are most often deployed in labs running standardized experimental processes in a production environment such as quality control, clinical tests, forensics, or core facilities providing standardized services like sequencing. The adoption of LIMS in a research and development (R&D) environment can be challenging because of the fluid nature of R&D experiments. The scope of their features varies greatly across vendors and products. They generally include different functional modules, including sample tracking modules, experimental data modules, equipment modules, ordering systems modules, dataflow management, and report generation modules, and the like.

A sample tracking module can be configured to, for example, help document, identify, and locate samples processed by a lab. Such a module makes it possible to define different categories of samples, associate data with the samples, and describe the sample content, and generate unique sample identification (ID) numbers that can be printed on, for example, bar codes. In addition, a sample tracking module includes a hierarchical model of the lab storage resources (e.g., freezers, liquid nitrogen, cold room), making it possible to associate a sample with a unique storage location.

A sample experimental data module can be configured to, for example, manage data produced by measurement instruments. In some cases, the LIMS server is connected to the instruments producing the data so that data are imported in the LIMS automatically as soon as they are generated.

An equipment module can be configured to, for example, list pieces of equipment, track their maintenance status, warranty, depreciation, and possibly their schedule.

An ordering system module can be configured to, for example, track the quantities of supplies and reagents used by the lab and help streamline orders of supplies.

A dataflow management and report generation module can be configured to, for example, capture standardized dataflows and generate analysis reports.

### Electronic Lab Notebooks (ELN)

A laboratory notebook, also known as a lab notebook, or lab journal, is a primary record of research. Researchers can use a lab notebook to document their hypotheses, experiments, and initial analysis or interpretation of these experiments. The notebook serves as an organizational tool, a memory aid, and can also have a role in protecting any intellectual property that comes from the research. However, there is no universal convention for keeping lab notebooks. They are generally permanently bound, and pages are numbered. Entries are written with a permanent writing tool. Lab notebook entries are generally organized by date and by experiment. They are supposed to be written as the experiments progress, rather than at a later date. In many laboratories, it is the original place of record of data as well as any observations or insights. For data recorded by other means (e.g., on a computer), the lab notebook will record that the data was obtained, and the identification of the data set will be given in the notebook. Many adhere to the concept that a lab notebook should be thought of as a diary of activities that are described in sufficient detail to allow another scientist to replicate the steps.

Historically, lab notebooks were used to support patent applications. However, in 2013 the United States (US) changed its patent law to award priority to the first person to file, rather than the first person to invent. In this context, the lab legal value of notebook is not as important as it used to be.

Paper notebooks are still used in many laboratories. As research became increasingly digital, the paper notebook was complemented by a nebula of data files on desktop computers, shared drives, cloud storage. Eventually, people got tired of the outdated paper notebook. Some attempted to replace the paper notebooks with various electronic documents. Others may have stopped keeping a lab notebook altogether, preferring to rely on raw data files. The proliferation of electronic resources used in research has disrupted record-keeping forever.

While the legal requirements to record research activities have eased off, record keeping is still necessary to ensure the reproducibility of research results. It is the ambition of electronic notebooks to bring some sanity back to the documentation of research activities by integrating disparate data into unified entries that are faster to write, easy to read, and convenient to search.

The full realization of the transition from physical paper to digital documentation means that there are more options to choose from than ever before. This category of products has not stabilized. Different vendors proposed fairly different solutions to the documentation of research activities.

Many of the ELNs are document-centric. To various degrees, they try to improve the experience of working with paper notebooks and provide various features to make the process faster, including experiment templates, libraries of protocols, or dataflow modules. At the end of the day, they give users a great deal of flexibility to edit the documents produced by the ELN system.

### Limitations of Electronic Laboratory Notebooks

However, keeping a lab notebook may be one of the most challenging aspects of scientific research. It's fair to say that the lab notebook has a bad reputation. It's often perceived as a waste of valuable time that would be better spent collecting more data. This perception is the direct consequence of the limitations of paper notebooks used until the lab notebooks started turning digital about 20 years ago.

The linear format of paper notebooks was not very suitable to keep track of multiple experiments conducted in parallel. For instance, someone could be working on assembling a plasmid while at the same time preparing a cell culture that will be transformed with the plasmid. A project may require working with mice and cell cultures at the same time. One solution to this challenge was to dedicate different notebooks to different aspects of a project or to different projects. However, there is so far this approach can go.

Papers notebooks are notoriously difficult to search. Flipping pages after pages of poorly handwritten notes trying to remember the details of an experiment could be very frustrating. This limitation of paper records challenges the value of keeping a notebook. What's the point of spending time documenting experiments if it proves virtually impossible to retrieve critical information when you need to?

The personal nature of paper notebooks makes collaborations difficult. For legal reasons, notebooks were assigned to a person, not to a group. Collaborative projects that require experiments performed by different persons become extremely difficult to track because records are scattered across multiple notebooks belonging to different collaborators.

Further, keeping paper notebooks is very time-consuming because the same protocols have to be written over and over. After a while, it becomes tedious to recopy the same transformation protocol every week. This prompts people to take shortcuts, take increasingly spotty notes until they reach a point where the notes captured in a paper notebook are essentially useless.

Accordingly, ELNs suffer from a number of limitations, including, for example, unstructured data, ability to capture large data sets, ability to track samples, ability to track computational steps of research steps, the ELN entries are unconstrained, etc. Below is a further look at these limitations.

1. ELNs include unstructured data: Because ELNs are document-centric, they make it very difficult to analyze data collected during the experiments they describe. Some of the data is embedded in the unstructured format in the document. Extracting data from lab notebook entries is next to impossible. Data is not structured in a way that makes it suitable for analysis as it would be if the data were organized in a database.

2. ELNs don't capture large datasets: Because ELNs are modeled after paper notebooks, they are not suitable to keep track of large datasets. While it is possible to copy and paste one picture or one reading of one instrument, the notebook format is just not adequate to record large datasets like sequencing reads, time series of spectrophotometric data, etc. As a result, electronic lab notebook struggle to capture the relation between the description of an experiment and the data produced by these experiments. The association takes the form of a file name and location or link to a file-sharing service. They are not required and can be broken.

3. ELNs don't track samples: Most experiments are physical operations that use and produce physical samples. Capturing the relation between the description of an experiment and the samples it uses and produces is challenging. Some ELNs include a LIMS and offer the possibility of linking LIMS records to ELN entries, but these links are optional and poorly formalized as the relation between the samples, and the experiment is not always properly articulated.

4. ELNs don't keep track of the computational steps of research steps: Today, experiments are complex processes that include computational operations and physical operations. Today's ELNs are unable to properly describe the computational aspects of an experiment.

5. ELN entries are unconstrained: ELN systems give a lot of flexibility to the user in the way they describe their experiments. While they allow users to take advantage of libraries of protocols and experiment templates that can save time spent keeping records, they enable users to modify content as in a diary.

6. ELN don't interact with automated instruments: Because ELNs have been designed to report data or experiment results, they are not able to interact with programmable instruments such as robotic liquid handlers, microscopes, electrophoresis capillary systems, and other computer-controlled instruments. It's therefore difficult to properly describe in a notebook format the complex protocols executed by these programmable instruments.

The network(s) 104 can include any appropriate network, including an intranet, the Internet, a cellular network, a local area network (LAN), or any other such network or combination, and communication over the network can be enabled via wired and/or wireless connections.

Accordingly, in accordance with various embodiments, a resource provider environment can be used to provide dataflow management services for managing laboratory operations and the like. The resource provider environment 106 can include any appropriate components for receiving requests and returning information or performing actions in response to those requests. As an example, resource provider environment 106 might include Web servers and/or application servers for receiving and processing requests, then returning optimized laboratory processes. While this example is discussed with respect to the internet, web services, and internet-based technology, it should be understood that aspects of the various embodiments can be used with any appropriate services available or offered over a network in an electronic environment.

In various embodiments, resource provider environment 106 may include various types of resources that can be utilized by multiple users or applications for a variety of different purposes. In at least some embodiments, all or a portion of a given resource or set of resources might be allocated to a particular user or allocated for a particular task, for at least a determined period of time. The sharing of these multi-tenant resources from a provider environment is often referred to as resource sharing, Web services, or “cloud computing,” among other such terms and depending upon the specific environment and/or implementation. Methods for enabling a user to reserve various resources and resource instances are well known in the art, such that a detailed description of the entire process, and explanation of all possible components, will not be discussed in detail herein. In this example, resource provider environment 106 includes a plurality of resources 114 of one or more types. These types can include, for example, application servers operable to process instructions provided by a user or database servers operable to process data stored in one or more data stores 116 in response to a user request.

In various embodiments, resource provider environment 106 may include various types of resources that can be utilized for providing dataflow management services for managing laboratory operations processing image data. In this example, resource provider environment 106 includes dataflow system 124 and training system 130. The systems may be hosted on multiple server computers and/or distributed across multiple systems. Additionally, the systems may be implemented using any number of different computers and/or systems. Thus, the systems may be separated into multiple services and/or over multiple different systems to perform the functionality described herein.

Dataflow system 124 is operable to specify, control, and document laboratory operations in, for example, research, quality control, forensics, diagnostics, etc. Dataflow system 124 can generate a body of data sufficient to achieve a goal, such as testing a scientific hypothesis in basic research or developing a new product in the industry, developing and executing a manufacturing process, performing a series of tests on a sample to characterize its properties for quality control, environmental monitoring, diagnostics, or forensics purposes, etc. Laboratory operations can involve the coordination of various activities, such as experiments, data analysis, supply chain transactions, etc. Experiments can include, for example, a series of physical operations performed in an organization's laboratories to produce data using manual labor or automated instruments. Data analysis can include sequences of computational steps used to plan experiments or analyze the data produced by experiments. Supply Chain Transactions can include outsourcing steps of research projects to external vendors who have capabilities not available in-house. Supply chain transactions include, for example, purchases of material and supplies but also purchase of scientific services (DNA sequencing) or manufacturing services (gene synthesis).

Training system 130 is operable to develop models of laboratory processes. For example, in an embodiment, training system 130 captures the hierarchical nature of laboratory processes to develop libraries of subprocesses that can be quickly linked to define new processes. In various embodiments, a unified data model is utilized, where the data used as input and output of these services define paths throughout this network of services, and each path corresponds to a different process. These services and processes can be defined using existing business process automation tools and other computational paradigms.

### Business Process Automation

In an embodiment, business processes are dataflows that involve manual steps, computational steps, and supply chain transactions. The evaluation of a mortgage application is a good example of a business process. It involves manual steps completed by different stakeholders, such as the application submitted by the applicant's or the risk analysis performed by the underwriter. It includes computational steps like retrieving the applicant credit report. It includes supply chain transactions such as property appraisal and inspection.

Business Process Automation (BPA) applications are software systems used to streamline business processes. They allow businesses to formalize their processes in custom programs that break down a process in individual steps. They make it possible to assign individual tasks to different categories of stakeholders and provide them with the data they need to perform the task. They can manage dependencies between tasks and assign tasks to project managers only when all the conditions necessary to perform the task are met. Some BPA systems allow users to define computational steps that can be executed either within the system itself or by calling a remote web service through an API. This possibility creates the possibility to connect a BPA system to a procurement system to include steps corresponding to supply chain transactions.

There are countless BPA systems on the market. Their capabilities vary greatly. Some offer little more than a task management system. Others allow users to describe dataflows but do not provide data management capabilities. BPA systems are also known as Business Process Management systems (BPM) or dataflow management systems. Enterprise Resources Planning systems (ERP) like SAP include BPA features.

It should be noted that there are fundamental differences between laboratory processes and business processes. For example, laboratory processes need to be developed. In an example, industry experts can specify business processes with assurance that they can provide the desired outcome. A banker, for example, can quickly outline the process to fulfill loan applications with a high level of confidence that loan applications will be processed successfully within a predictable timeframe. This type of assurance is unknown when processes involve laboratory operations in life sciences, chemical engineering and related fields. Experts can specify a sequence of operations that might give the desired outcomes, but the actual performance of the process needs to be tested to determine the process outcome and performance empirically. Because experts cannot anticipate the performance of a process, most laboratory processes are the results of an extensive process development effort that can take years and cost millions. In biomanufacturing (a mature industry), the development of a process to manufacture a new drug candidate will take six to 12 months with costs in the range of $2-5M. For example, the development of a kit to diagnose a viral infection like COVID-19 can take months and millions before a diagnostic procedure and the supporting kit is sufficiently affordable and robust to be made broadly available to the healthcare system. In drug discovery and basic research, it is not uncommon to spend years developing all the steps of an experiment. When a robust process is available, it is applied to a number of cases to generate a dataset large enough to reach robust conclusions or to make several batches of a product.

In another example, laboratory processes have a high rate of systemic failure. In an example, so much process development is needed because laboratory processes have a high rate of deterministic failures. The same operation applied to different cases have different outcomes. For example, a process to synthesize a gene will work with 90% of genes but will fail with 10% of the genes because their sequences have unknown properties that make them incompatible with this manufacturing process. Most failures in traditional production processes are random errors that can be addressed by simple rework strategies based on the repetition of the failed step. The prevalence of systematic errors greatly increases the complexity of laboratory processes as they require to specify alternative processes that may circumvent the cause of failure in executing the original process.

In yet another example, identical outcomes can be achieved by very different laboratory processes. In an example, in many situations, many alternative processes can be considered to achieve the same outcome (production of a pure protein, synthesis of a gene, collection of a dataset). While these different processes may have the same endpoint, their performance (delay, costs, labor, success rate) may be very different in ways that are not possible to predict.

The consequence of these challenges is that life scientists and people working in biotechnology need to be able to define many complex processes to test them, compare their performance, before settling down on a process that meets their requirements. Most of these process variants will be executed on a limited number of cases to evaluate their performance. The adhoc process modeling approach used to model business processes is not suitable to support the development of laboratory processes. Even using modern process automation tools, implementing a new process takes too much time and costs too much when it is necessary to test 100s of process variants on a small number of cases.

It should be noted that the challenge of developing complex processes is not limited to life scientists working in laboratories and the techniques described herein may be used for a wide variety of situations. For example, techniques can include R&D projects that might take place outside of a laboratory such as plant breeding programs that involve activities taking place in a greenhouse or in experimental plots. Rapid development of R&D process can also occur in other industries. For example, the manufacturing of race cars is conceptually similar to the development of biological experiment, as is food processing and indeed operations in the dining industry It requires rapidly specifying different manufacturing processes to produce different prototypes and comparing their performance on the track. These processes will involve a number of computational steps, on site manufacturing operations, and supply chain transactions. Generally, any industry that need to rapidly iterate complex cyberphysical processes can benefit from approaches described herein.

In various embodiments, the resources 114 can take the form of servers (e.g., application servers or data servers) and/or components installed in those servers and/or various other computing assets. In some embodiments, at least a portion of the resources can be “virtual” resources supported by these and/or components. While various examples are presented with respect to shared and/or dedicated access to disk, data storage, hosts, and peripheral devices, it should be understood that any appropriate resource can be used within the scope of the various embodiments for any appropriate purpose, and any appropriate parameter of a resource can be monitored and used in configuration deployments.

In at least some embodiments, an application executing on the client device 102 that needs to access resources of the provider environment 106, for example, to manage dataflow system 124 and/or training system 130, implemented as one or more services to which the application has subscribed, can submit a request that is received to interface layer 108 of the provider environment 106. The interface layer 108 can include application programming interfaces (APIs) or other exposed interfaces, enabling a user to submit requests, such as Web service requests, to the provider environment 106. Interface layer 108, in this example, can also include a scalable set of customer-facing servers that can provide the various APIs and return the appropriate responses based on the API specifications. Interface layer 108 also can include at least one API service layer that, in one embodiment, consists of stateless, replicated servers that process the externally-facing customer APIs. The interface layer can be responsible for Web service front-end features such as authenticating customers based on credentials, authorizing the customer, throttling customer requests to the API servers, validating user input, and marshaling or un-marshaling requests and responses. The API layer also can be responsible for reading and writing database configuration data to/from the administration data store, in response to the API calls. In many embodiments, the Web services layer and/or API service layer will be the only externally visible component or the only component that is visible to and accessible by customers of the control service. The servers of the Web services layer can be stateless and scaled horizontally, as known in the art. API servers, as well as the persistent data store, can be spread across multiple data centers in a region, for example, such that the servers are resilient to single data center failures.

When a request to access a resource is received at the interface layer 108 in some embodiments, information for the request can be directed to resource manager 110 or other such systems, service, or component configured to manage user accounts and information, resource provisioning and usage, and other such aspects. Resource manager 110 can perform tasks such as to communicate the request to a management component or other control component which can manage distribution of configuration information, configuration information updates, or other information for host machines, servers, or other such computing devices or assets in a network environment, authenticate an identity of the user submitting the request, as well as to determine whether that user has an existing account with the resource provider, where the account data may be stored in at least one data store 112 in the resource provider environment 106. The resource manager can, in some embodiments, authenticate the user in accordance with embodiments described herein based on voice data provided by the user.

A host machine 120 in at least one embodiment can host the dataflow system 124 and training system 130. It should be noted that although host machine 120 is shown outside the provider environment, in accordance with various embodiments, dataflow system 124 and training system 130 can both be included in provider environment 106, while in other embodiments, one or the other can be included in the provider environment. In various embodiments, one or more host machines can be instantiated to host such systems for third-parties, additional processing of preview requests, and the like.

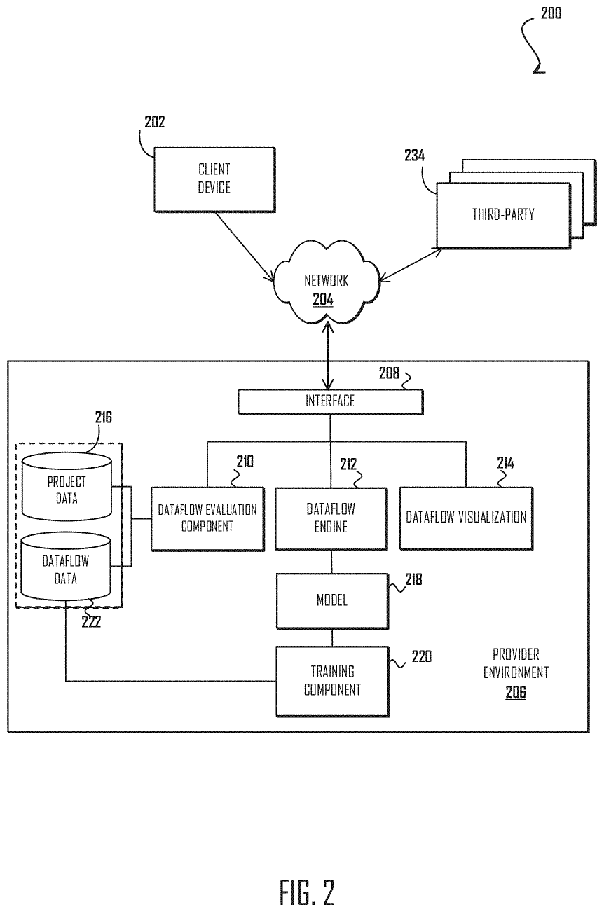

FIG. 2 illustrates an example environment 200 in which aspects of the various embodiments can be implemented. It should be understood that reference numbers are carried over between figures for similar components for purposes of simplicity of explanation, but such usage should not be construed as a limitation on the various embodiments unless otherwise stated. In this example, a user can utilize a client device 202 to communicate across at least one network 204 with a resource provider environment 206. The client device 202 can include any appropriate electronic device operable to send and receive requests or other such information over an appropriate network and convey information back to a user of the device. The devices may include, for example, any suitable combination of components that operate to create, manipulate, access, and/or transmit project information. Examples of such client devices 202 include personal computers, tablet computers, smartphones, notebook computers, an electronic notebook, and the like.

The user can include, for example, various project members (e.g., engineers, developers, management, administrators, operators, etc.) that generate project information while working on a laboratory project. Project members may generate a wide variety of project information, including project requirements information (e.g., project milestones, milestone deadlines, expected project deliverables, etc.) and project assumptions information (vendor performance estimates, vendor delivery time estimates, project member availability, etc.) Project members can include, for example, any party involved in setting requirements for a project, completing tasks for a project, or performing any other appropriate functions associated with the project.

Project information can include, for example, any information related to a laboratory project. As examples, project information may include, in certain embodiments, general project information, project cost estimates, application impact information, infrastructure impact information, project requirements, project assumptions, project savings, project history, project contacts, among others. Other project information may include, for example, start point and end point, wherein the start point represents that data models that may be available to a researcher or a scientist. The end point may represent a data model that may prove a hypothesis, disprove a hypothesis and/or mark completion of a project. Project information may further include requirements such as cost, speed of competition, available lab equipment, time of delivery, data-in and data-out compatibility, approved vendors, etc.

The network(s) 204 can include any appropriate network, including an intranet, the Internet, a cellular network, a local area network (LAN), or any other such network or combination, and communication over the network can be enabled via wired and/or wireless connections.

The resource provider environment 206 can include any appropriate components for receiving requests and returning information or performing actions in response to those requests. As an example, resource provider environment 206 might include web servers and/or application servers for receiving and processing requests, then returning laboratory dataflows or other such content or information in response to the request. While this example is discussed with respect to the internet, web services, and internet-based technology, it should be understood that aspects of the various embodiments can be used with any appropriate services available or offered over a network in an electronic environment.

Resource provider environment 206 can include dataflow engine 212, dataflow evaluation component 210, dataflow visualization component 214, training component 220, and model 218, although additional or alternative components and elements can be used in such a system in accordance with the various embodiments. Accordingly, it should be noted that additional services, providers, and/or components can be included in such a system, and although some of the services, providers, components, etc., are illustrated as being separate entities and/or components, the illustrated arrangement is provided as an example arrangement and other arrangements as known to one skilled in the art are contemplated by the embodiments described herein.

Client device 202 can be utilized by a project manager to send project information to dataflow evaluation component 210 over network 204 to be received at an interface 208 and/or networking layer of resource provider environment 206. The interface and/or networking layer can include any of a number of components known or used for such purposes, as may include one or more routers, switches, load balancers, web servers, application programming interfaces (APIs), and the like.

Interface 208 can be configured to receive specific types of project information, and the project information can be aggregated and stored in data store 216. As described, aggregating project information in a centralized location (e.g., data store 216) may allow the system to conserve memory and bandwidth over a system in which pieces of project information are stored in different locations, and in different formats across the enterprise and must be located and retrieved from these various locations during an export operation.

Dataflow evaluation component 210 can include one or more processing components operable to perform various functions to aggregate, group, store, format, and export project information. For instance, in some embodiments, dataflow evaluation component 210 may receive and aggregate project information from project members in one or more databases such as data store 216 or other such repository. For instance, in certain embodiments, dataflow evaluation component 210 may receive an indication from a project member of a type of project information that the project member desires to input. In response, dataflow evaluation component 210 collects the project information from the project member.

Upon receiving project information, dataflow evaluation component 210 may, in certain embodiments, logically groups certain information together for storage in data store 216, for example. For instance, dataflow evaluation component 210 may select certain project information received from a project member and group that information with project information received from another project member. Dataflow evaluation component 210 may group information based upon any appropriate criteria, including the type of project information at issue or the time when project information is received. As an example, many laboratories can produce millions of samples a year. An essential aspect of managing these samples is tracking their location at all times. Considering the diversity of storage equipment and facilities, it is difficult to get a good data model of storage locations.

Embodiments described herein can utilize a hierarchical model in which each storage location can be contained within another location, has a capacity, and an occupation (occupation is the number of samples at the location, it should not exceed the location capacity).

In various embodiments, dataflow engine 212 may detect a triggering event associated with particular project information and/or project parameters and, in response, gather appropriate project information to be formatted and transmitted to an appropriate entity. In general, a triggering event may comprise any signal or event that indicates to dataflow engine 212 that certain project information should be gathered and transmitted to a particular entity. As an example, in certain embodiments, a triggering event may include a request for certain project information. As another example, a triggering event may occur upon the completion of particular project milestones or after the passage of a certain amount of time from the commencement of the project.

To perform these tasks, in certain embodiments, dataflow engine 212 may select and operate according to one or more dataflows and/or processes. Dataflows may contain instructions that instruct dataflow engine 212 as to what operations should be performed with respect to certain project information. This can include, for example, a series of tasks executed by a single project member, generally within a day of work. A dataflow in certain embodiments does not include decision points. A dataflow may be ordered by the manager of the project member executing the dataflow. The proper execution of the dataflow may be approved by the manager upon review of the data collected during the dataflow execution. An example dataflow can include running a bioinformatics script to calculate the sequence of PCR primers, placing an order for a synthetic gene, starting a cell culture, extracting DNA from a cell culture.

A process, in various embodiments, may include a combination of dataflows executed by multiple project members over an extended period of time. Processes include multiple decision points that can lead to multiple execution paths. An example process can include developing a new gene therapy, making a library of 1000 yeast mutants, developing a manufacturing process to produce a drug, a paternity test based on DNA sequence analysis.

The dataflows can be associated with one of a number of categories. The categories can include, for example, laboratory dataflows, computational dataflows, supply chain dataflows.

Laboratory dataflows are typically performed in a user's laboratory by the user personnel. They include step-by-step instructions to manipulate samples and collect data. Laboratory dataflows are the kind of dataflows typically handled by current LIMS and ELN. Examples of laboratory dataflows include cell culture dataflows, extraction dataflows, purification dataflows, quality control dataflows, enzymatic reactions to assemble DNA molecules, etc.

As to computational dataflows, many laboratory processes involve computational steps that are performed before experimental dataflows are performed or after they have been executed. Computational steps that come ahead of experimental dataflows or supply chain dataflows are typically used to calculate aspects of the experimental dataflows. For example, it may be necessary to design a small DNA molecule (a primer) that will then be ordered from a supplier and used to amplify a DNA fragment. Most laboratory processes will end up with a computational step that will analyze the data collected in the lab. For example, DNA sequencing data are not directly usable. They need to be analyzed for specific purposes by specialized bioinformatics programs in order to provide the answer that the laboratory process aims to provide.

While computational dataflows are an integral part of laboratory processes, they typically cannot be captured by conventional LIMS and ELN products because these systems are unable to manage large datasets that these sets typically require. They may also not have access to the numerous parameters that control the execution of computational steps. Finally, they do not have access to information regarding the server configuration and the version of the software used to perform the computational steps. Embodiments described herein include a library of computational services that can be used at different steps of laboratory processes. These applications run on specialized servers and can communicate with other applications through one or more application programming interfaces (APIs).

Rather than relying on project members copying and pasting data manually from one application to another, embodiments herein include automated dataflows that call these services from a business process automation environment through their API. This makes it possible to automatically send input data to these services and collect their output without manual intervention. For example, dataflows need data as input (i.e., project information) and produce data as output. Inputs/Outputs (IO) are records matching complex data types that represent both physical samples and the properties of these samples. Inputs can be provided through forms by the project members, and outputs can be returned to the project member as a report. However, I/O can also be accessed programmatically. This makes it possible to call dataflows from processes. A process can take input data from the project members. This input data can be processed to provide input data to dataflows included in the process. The dataflow can then return output data to the process. The output data can then be processed and passed as input to the next dataflow in the process until, eventually, the process returns the output data of the project member. In an example, the goal of an experiment may be to design a yeast strain with superior fermentation properties. This experiment is the process at the top of the abstraction hierarchy.

Supply chain dataflows can correspond to operations that are outsourced. Two categories of supply chain operations can be distinguished: the ordering of supplies used by laboratory dataflows and ordering of contract research or manufacturing services.

Automating the ordering of supplies is important to minimize variability that may result from ordering “similar” supplies rather than the ones that have been validated. Many of these supplies have limited shelf-lives, are expensive, and require a significant lead time. As a result, ordering needs to be carefully aligned with needs. In addition, specific information such as lot number or concentration about items received from suppliers and contractors needs to be captured in the systems in order to track their possible impact on the outcome of dataflows and processes using these items.

Automating the ordering of contract services is important because these are often complex orders that require communicating significant amounts of data to the suppliers. Acceptance of the orders is not automatic, and delivery can be somewhat unpredictable. Many laboratory processes can be held back for weeks until these services have been delivered. Errors in the ordering process may result in significant delays and financial losses.

In addition, project members can often combine the services of contractors in different ways to achieve similar results. Comparing the prices and delays of the different alternatives can be difficult.

Supply chain dataflows formalize the services provided by contractors and integrate them into laboratory dataflows. Depending on the data services provided by the vendor, the supply chain dataflows may communicate directly to the vendor information system through the vendor API or simply prepare orders that will be placed manually.

Dataflow engine 212 can utilize process interfaces to enforce data type checking. This ensures that data passed from a process to another process or dataflow are compatible. It is often necessary to retrieve all the data related to a particular process execution. Processes can include a CaseID or other identifier in their data model that is used as a common thread throughout the process execution. That makes it possible to retrieve all the data generated at different stages of the dataflow in relation to a particular case. In an example, a CaseID can be used to associate the sequencing data of a yeast strain with the fermentation performance of this strain. Ensuring the integrity of data collected throughout a laboratory process is something that conventional LIMS don't do well. Rather, they build data silos corresponding to individual dataflows, but they fail to associate data points across data stores.

The CaseID can be a process execution identifier. Embodiments herein also include a ProcessID (e.g., process identifier) that is unique to the process template. In various embodiments, when a process is revised, it gets a new ProcessID that can be used to retrieve data related to a particular process version.

Dataflow engine 212 is configured to use a data flow execution engine. Dataflow programming is a programming paradigm that models a program as a directed flow graph of the data flowing between nodes representing operations. Traditional programs such as workflow management system describe series of operations happening in a specific order. They emphasize commands that manipulate data in sequence. In contrast, dataflow programs emphasize the movement of data and represents programs as a series of connections between data streams. Explicitly defined inputs and outputs connect operations that can be executed as soon as all inputs become available. Thus, dataflow execution is inherently parallel and well adapted to deployment in scalable decentralized architectures. In addition to data availability, process execution is determined by decision points that use data collected during the execution of a process or dataflow and determines if the process execution was successful or not. There are two levels of decisions. Pass/Fail decisions at the level of the dataflow or process level and Pass/Fail decision at the case level. Control cases are processed to determine the success of a particular dataflow or process. When a dataflow or process fails, all the cases processed at the same time (same batch) are automatically failed. For example, if growth media are not sterile, all cell cultures started with the growth medium will have to be discarded. When a dataflow or process passes, then Pass/Fail decisions are made on a case by case level. Decisions points do not control the execution of the process as in rule-based process automation. Instead, decision points control the type of data output by the process which in turn become available as inputs for other processes.

In some cases, the pass/fail decision can be automated because it is based on simple data that can be collected with great accuracy (presence of contaminants in a growth media, the concentration of a DNA solution). In other cases, the pass/fail decision is made by an expert who will review the data collected during the process execution. There are situations when the experience is necessary to interpret ambiguous data.

In some cases, the course of action after a pass/fail decision can be determined by project managers who may be offered to choose possible alternative courses of action while respecting data type compatibility rules. If a dataflow or process fails, all cases processed during the process execution will have to be reprocessed. If the process passes but some cases fails, then the case may be reworked (the case goes back to the last passed stage of the process) or may be handled using an alternative strategy.

Dataflow process execution increase process reproducibility by eliminating subjective decisions regarding the sequence of dataflows and processes. Different cases may follow different paths through the process, but their path is driven by rules and runtime data instead of being driven by subjective decisions.

### Automated Processes

For example, at any point in time, a laboratory state can be described by data describing the state of cases going through the laboratory process. The state of the laboratory automatically determines what could be performed next. The sequence of tasks corresponding to the execution of a particular process will be determined by a number execution policies including:

First-in first out policies: Task execution can be enabled by the availability of the corresponding input data. The system maintains a queue of tasks assigned to groups of users qualified to perform them. Users pick tasks on a first come first served basis.

Process scheduling can be characterized in accordance with several aspects:
(i) Task prioritization: some tasks may be placed higher in the queue (rush orders that paid a premium, priority projects)
(ii) Batch structures: some tasks are best performed in batches because of fixed costs associated with a batch. In this case, the task will be executed only when the queue has reached a minimum size to fill a batch.
(iii) Timing constraints: some tasks need to be executed within a specific time frame. In this case they are only available to operators during this time frame.

Supply chain management: The execution of a process may start by reserving supplies or ordering and waiting for the delivery of necessary materials and supplies prior to starting laboratory operations.

Laboratory management daemons: A number of housekeeping processes can be put in place that trigger automatic actions when certain conditions are met: a list of reagents and samples that have passed their expiration date can be computed weekly, and lab technicians can be required to dispose of them. Supplies can be ordered automatically when their quantity goes under a critical threshold. Equipment can be calibrated on a regular basis.

### Laboratory Automation

In accordance with various embodiments, laboratory automation can refer to the deployment of computer-controlled instruments that automate physical operations such as liquid handling systems or high-throughput measurement instruments. Many laboratories have wasted millions of dollars in automated instruments without considering how these instruments will interact with the rest of the laboratory operations. Programmable instruments support high throughput of operations. To maximize their value, it is necessary to provide them with a lot of input samples, and it is necessary to be able to manage the data and samples they generate. It is not possible to automate physical operations without automating data flows. However, when data flows are automated, automated instruments are not conceptually different than human operators. They need to be provided instructions in a different format than human operators, but their contribution to over the overall process is exactly the same.

In an embodiment, automated laboratory management makes it possible to generate various reports that are not available to conventional LIMS. For example, users or third-parties 234 may desire to view aspects of the project information and can submit a request to view the project information. Dataflow visualization component 214 can obtain the requested project information and provide the project information in a format appropriate for the requesting party. Dataflow visualization component 214 may operate in conjunction with dataflow engine 212 to format and export certain project information to one or more appropriate entities as directed by dataflow engine 212 and dataflows. For instance, dataflow visualization component 214 may format certain laboratory information from project information in spreadsheet format and export the laboratory information to the appropriate entity.

In an embodiment, the reports can include process-level reports, case-level reports, dataflow-level reports, laboratory-level reports. Examples of process-level reports include operational reports such as the distribution of cases at different stages of the process, running costs, failure rate of cases going through the process, expected completion date. For processes designed to process a large number of related cases that will generate one dataset, it is possible to generate multi-dimensional datasets that associated all the data collected for individual cases and the preliminary analysis of these data.

Case-level reporting includes reports that provide extensive documentation of the operations and data collected in relation to a single case. This type of report could be particularly valuable in relation to regulated activities such as seeking regulatory approval of a product or manufacturing process, clinical diagnostics, or environmental monitoring operations. Reports such as success rate or cost of specific dataflows could be used to support a data-driven process improvement processes or justify investment in automated equipment.

Laboratory-level reports can include reports associated with project managers or equipment. For example, it can be interesting to generate the success rate of dataflows executed by individual project managers to identify project managers who may need new training. Similar reports could be generated for equipment to detect equipment that needs servicing.

In an embodiment, a model 218 can be trained using, for example, training component 220 on various models of laboratory processed in database 222. Training component 220 can learn various combinations or relations of features of laboratory processes, such that when particular project information is received as an input to the system, model 218 can be used to evaluate the project information to recognize the features and output the appropriate information to generate a laboratory process. Examples models include, for example, logistic regression, Naïve Baye, random forest, neural network, or support vector machines (SVMs), convolutional recurrent neural network, deep neural network, or other types of neural networks or models, and/or combination of any of the above models, stacked models and heuristic rules. Various other approaches can be used as well as discussed and suggested elsewhere herein.

In accordance with various embodiments, the various components described herein may be performed by any number of server computing devices, desktop computing devices, mainframe computers, and the like. Individual devices may implement one of the components of the system. In some embodiments, the system can include several devices physically or logically grouped to implement one of the modules or components of the message service. In some embodiments, the features and services provided by the system may be implemented as web services consumable via a communication network. In further embodiments, the system is provided by one more virtual machines implemented in a hosted computing environment. The hosted computing environment may include one or more rapidly provisioned and released computing resources, which computing resources may include computing, networking and/or storage devices. A hosted computing environment may also be referred to as a cloud computing environment.

### Automated Process Automation

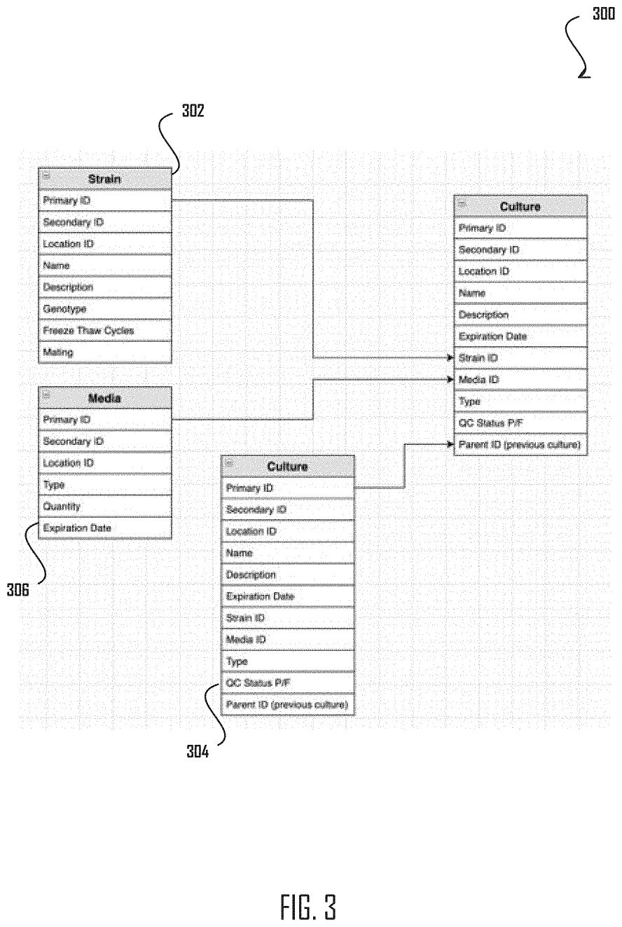

FIG. 3 illustrates example 300 of a unified data model in accordance with various embodiments. As described, the data used as input and output of various services define paths throughout a network of services, and each path can correspond to a different process. In an embodiment, at a high-level, laboratory processes can transform physical samples and collect data on these samples. For example, a DNA extraction dataflow can create a DNA solution sample out of a cell culture sample. Passing the DNA solution in an analytical instrument (spectrophotometer, capillary electrophoresis) can produce data associated with the DNA solution sample. Traditional LIMB rely on one size fits all data model of physical samples and cannot associate data with these samples. That makes it very difficult to analyze data because there is no build-in link between samples and measurement values.

In accordance with various embodiments, a dataflow-centric data model that integrates the description of the physical sample and the data collected on these physical samples can be used. In such an approach, there is no conceptual difference between samples and data. The data model is dictated by the information needed to execute tasks and the information these tasks produced. For example, a task aiming at starting a cell culture will typically need a strain of cells 302 used to inoculate culture 304 and growth medium 306 as input. If the culture is derived from a previous culture, then it also needs to capture this information. The culture starting task can create a sample of the type cell culture to which may be attached a Pass/Fail quality control flag. In this context, the growth media 306, is considered a sample, which is not something most LIMS would handle as this type of preparation is generally not tracked. Media preparation samples would be produced by the media preparation task.

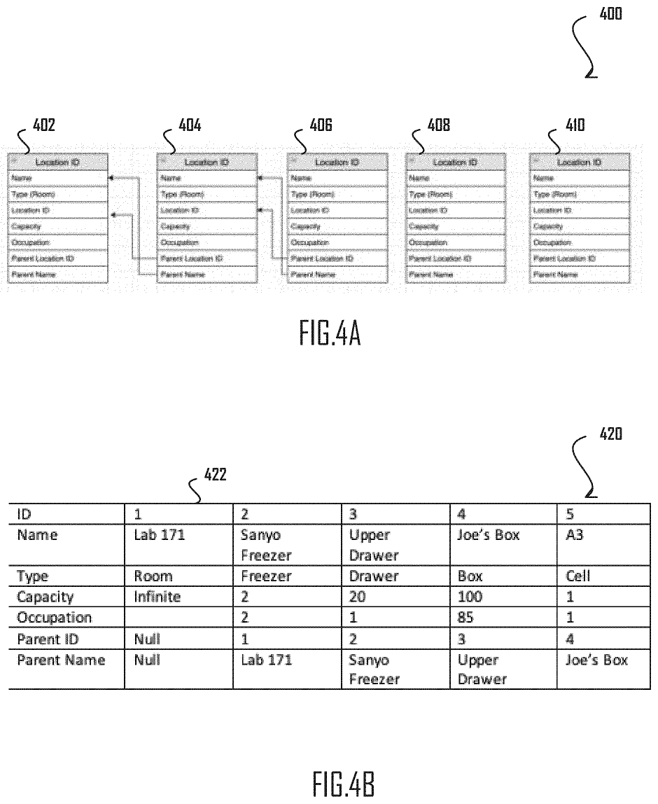

FIGS. 4A and 4B illustrate example 400 of a data model to track a sample in accordance with various embodiments. Most laboratories can produce millions of samples a year. An essential aspect of managing these samples is to track their location at all times. Considering the diversity of storage equipment and facilities, it is difficult to get a good data model of storage locations. Embodiments described herein rely on a hierarchical model in which each storage location is contained within another location, has a capacity and an occupation (occupation is the number of samples at the location, it should not exceed the location capacity). For example, FIG. 4A illustrates storage locations 402, 404, 406, 408, and 410. As shown, the hierarchical model indicates that each storage location is contained within another location, whether a storage location has a capacity, and an occupation of each storage location. Example 420 of FIG. 4B illustrates table 422, which includes information related to each storage location shown in FIG. 4A. As illustrated, the information corresponds to a name, type of location, capacity, occupation, parent ID, and parent name. This information can be used to, for example, track the location of a sample. In accordance with various embodiments, the hierarchical model of storage locations can be applied to other situations including, for example, tracking plants in experimental fields or physical parts in a warehouse.

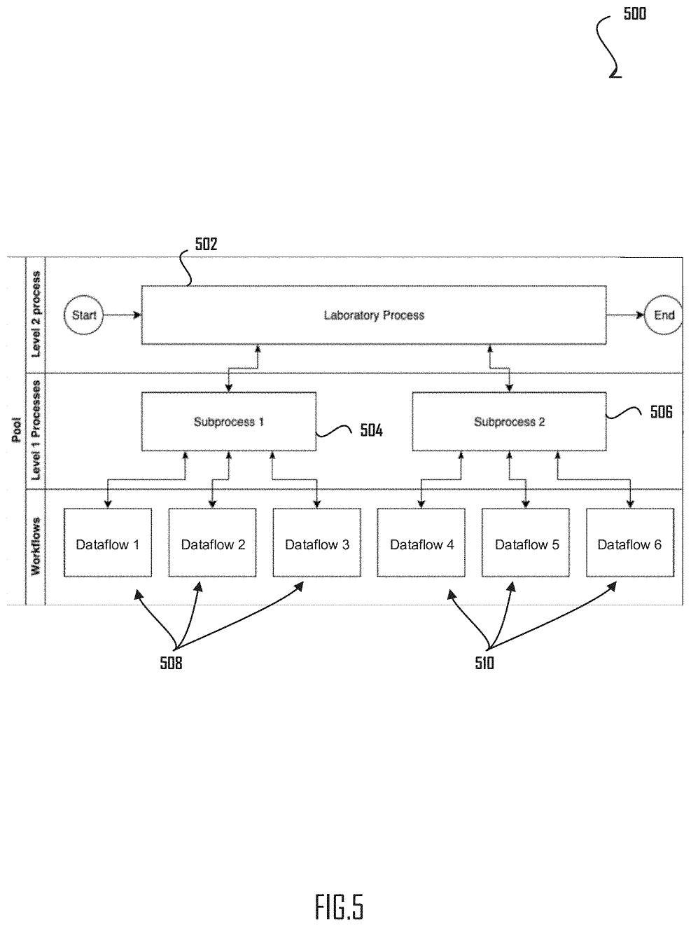

FIG. 5 illustrates example 500 of programmatic access to inputs and outputs in accordance with an embodiment. As described, tasks need data as input and produce data as output. Inputs/Outputs (TO) are records matching complex data types that represent both physical samples and the properties of these samples. In this example, the goal of an experiment may be to design a yeast strain with superior fermentation properties. This experiment is represented by process 502 at the top of the abstraction hierarchy. Process 502 includes two subprocesses: engineering of the yeast strain 504 and testing of the engineered yeast strain 506. These are two subprocesses of the highest-level process. Engineering the yeast strain will call dataflow 508 describing basic tasks such as growing the parent yeast strain, preparing the DNA to be inserted in the yeast strain, selecting mutant strains, and verifying selected mutants. The testing process will include dataflows 510, aiming at measuring the growth of the engineered yeast strain in the presence of a particular feedstock and measuring the chemical composition of the growth media after the fermentation.

Advantageously, data type compatibility and hierarchical process definition accelerates process development. For example, a variant of a laboratory process can be quickly created by substituting subprocess 1 504 with another subprocess with compatible data types. Similarly, variants of subprocess 1 504 can be created by inserting various variants of dataflow 1 that share common input and output data types. Process variants can be created manually by letting the process designer substitute a subprocess or a dataflow with another one with compatible datatypes. Alternatively, it possible to automate the generation of process variants by providing a process template and testing all possible valid combinations of subprocesses and dataflows available in a library. In another embodiment, the development of a process can bel automated without providing a process template.

In an embodiment, the dataflows and subprocesses of FIG. 5 can be offered as services that broadcast on a computer network the type of their input data along with information about their internal state including their availability, expected execution time, and any other relevant properties. Some of these properties such as the type of input data is static while other information such as expected execution is dynamic. For example, a service corresponding to a supply chain transaction may broadcast different fulfillment times and possibly different prices based on the current state of its order book. A process can monitor the state of this grid of services to build routing tables based on data type compatibility. User requests defined by the type of their input and output data can be submitted to a router that will dynamically determine the optimal process as a path through the grid of services connecting input and output.

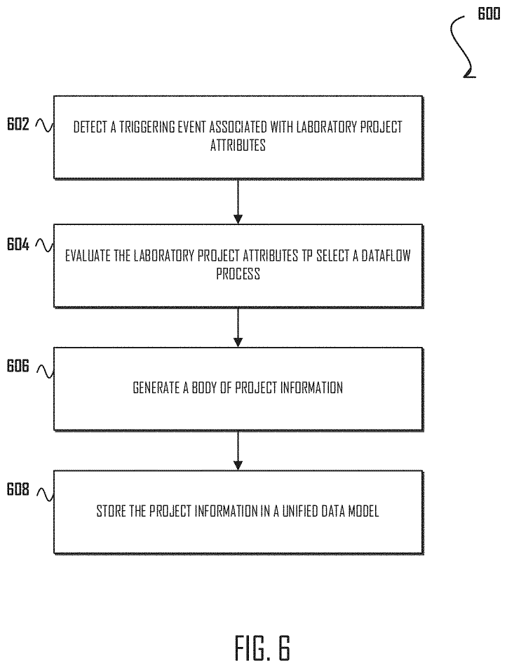

FIG. 6 illustrates an example process 600 for obtaining project information for a laboratory project in accordance with various embodiments. It should be understood that, for any process discussed herein, there can be additional, fewer, or alternative steps, performed in similar or different orders, or in parallel, within the scope of the various embodiments unless otherwise stated. In this example, a triggering event associated with laboratory project attributes is detected 602. The laboratory project attributes can be evaluated 604 with a trained model to select a dataflow process. The dataflow process can be used to generate 606 a body of project information based on the dataflow process, wherein the project information is used to test a scientific hypothesis. Thereafter, the project information can be stored 608 in a unified data model.

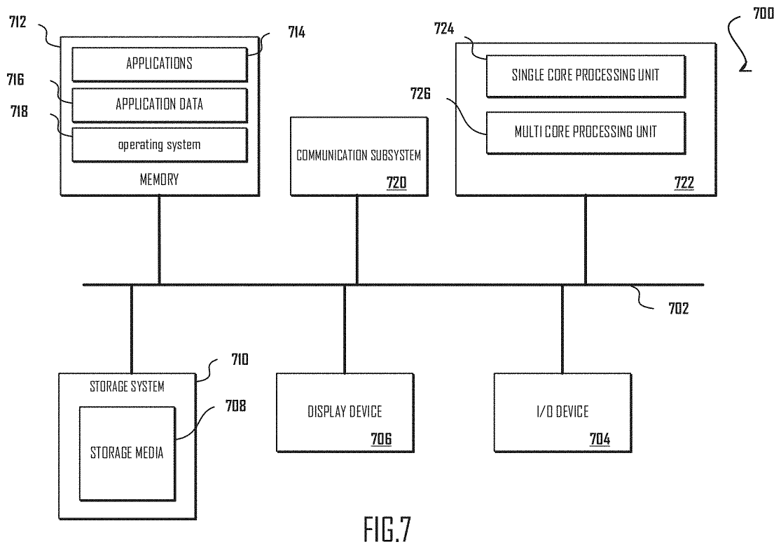

FIG. 7 shows an example computer system 700, in accordance with various embodiments. In various embodiments, computer system 700 may be used to implement any of the systems, devices, or methods described herein. In some embodiments, computer system 700 may correspond to any of the various devices described herein, including, but not limited, to mobile devices, tablet computing devices, wearable devices, personal or laptop computers, vehicle-based computing devices, or other devices or systems described herein. As shown in FIG. 7, computer system 700 can include various subsystems connected by a bus 702. The subsystems may include an I/O device subsystem 704, a display device subsystem 706, and a storage subsystem 710, including one or more computer-readable storage media 708. The subsystems may also include a memory subsystem 712, a communication subsystem 720, and a processing subsystem 722.

In system 700, bus 702 facilitates communication between the various subsystems. Although a single bus 702 is shown, alternative bus configurations may also be used. Bus 702 may include any bus or other components to facilitate such communication as is known to one of ordinary skill in the art. Examples of such bus systems may include a local bus, parallel bus, serial bus, bus network, and/or multiple bus systems coordinated by a bus controller. Bus 702 may include one or more buses implementing various standards such as Parallel ATA, serial ATA, Industry Standard Architecture (ISA) bus, Extended ISA (EISA) bus, MicroChannel Architecture (MCA) bus, Peripheral Component Interconnect (PCI) bus, or any other architecture or standard as is known in the art.

In some embodiments, I/O device subsystem 704 may include various input and/or output devices or interfaces for communicating with such devices. Such devices may include, without limitation, a touch screen or other touch-sensitive input device, a keyboard, a mouse, a trackball, a motion sensor or other movement-based gesture recognition device, a scroll wheel, a click wheel, a dial, a button, a switch, audio recognition devices configured to receive voice commands, microphones, image capture based devices such as eye activity monitors configured to recognize commands based on eye movement or blinking, and other types of input devices. I/O device subsystem 704 may also include identification or authentication devices, such as fingerprint scanners, voiceprint scanners, iris scanners, or other biometric sensors or detectors. In various embodiments, I/O device subsystem may include audio output devices, such as speakers, media players, or other output devices.

Computer system 700 may include a display device subsystem 706. Display device subsystem may include one or more lights, such as one or more light emitting diodes (LEDs), LED arrays, a liquid crystal display (LCD) or plasma display or other flat-screen display, a touch screen, a head-mounted display or other wearable display device, a projection device, a cathode ray tube (CRT), and any other display technology configured to visually convey information. In various embodiments, display device subsystem 706 may include a controller and/or interface for controlling and/or communicating with an external display, such as any of the above-mentioned display technologies.

As shown in FIG. 7, system 700 may include storage subsystem 710 including various computer-readable storage media 708, such as hard disk drives, solid-state drives (including RAM-based and/or flash-based SSDs), or other storage devices. In various embodiments, computer-readable storage media 708 can be configured to store software, including programs, code, or other instructions, that is executable by a processor to provide the functionality described herein. In some embodiments, storage subsystem 710 may include various data stores or repositories or interface with various data stores or repositories that store data used with embodiments described herein. Such data stores may include, databases, object storage systems and services, data lakes or other data warehouse services or systems, distributed data stores, cloud-based storage systems and services, file systems, and any other data storage system or service. In some embodiments, storage subsystem 710 can include a media reader, card reader, or other storage interfaces to communicate with one or more external and/or removable storage devices. In various embodiments, computer-readable storage media 708 can include any appropriate storage medium or combination of storage media. For example, computer-readable storage media 708 can include, but is not limited to, any one or more of random-access memory (RAM), read-only memory (ROM), electronically erasable programmable ROM (EEPROM), flash memory or other memory technology, optical storage (e.g., CD-ROM, digital versatile disk (DVD), Blu-ray® disk or other optical storage device), magnetic storage (e.g., tape drives, cassettes, magnetic disk storage or other magnetic storage devices). In some embodiments, computer-readable storage media can include data signals or any other medium through which data can be transmitted and/or received.

Memory subsystem 712 can include various types of memory, including RAM, ROM, flash memory, or other memory. Memory subsystem 712 can include SRAM (static RAM) or DRAM (dynamic RAM). In some embodiments, memory subsystem 712 can include a BIOS (basic input/output system) or other firmware configured to manage initialization of various components during, e.g., startup. As shown in FIG. 7, memory subsystem 712 can include applications 714 and application data 716. Applications 714 may include programs, code, or other instructions, that can be executed by a processor. Applications 714 can include various applications such as browser clients, campaign management applications, data management applications, and any other application. Application data 716 can include any data produced and/or consumed by applications 714. Memory subsystem 712 can additionally include operating system 718, such as macOS®, Windows®, Linux®, various UNIX® or UNIX- or Linux-based operating systems, or other operating systems.

System 700 can also include a communication subsystem 720 configured to facilitate communication between system 700 and various external computer systems and/or networks (such as the Internet, a local area network (LAN), a wide area network (WAN), a mobile network, or any other network). Communication subsystem 720 can include hardware and/or software to enable communication over various wired (such as Ethernet or other wired communication technology) or wireless communication channels, such as radio transceivers to facilitate communication over wireless networks, mobile or cellular voice and/or data networks, Wi-Fi networks, or other wireless communication networks. Additionally, or alternatively, communication subsystem 720 can include hardware and/or software components to communicate with satellite-based or ground-based location services, such as GPS (global positioning system). In some embodiments, communication subsystem 720 may include, or interface with, various hardware or software sensors. The sensors may be configured to provide continuous or and/or periodic data or data streams to a computer system through communication subsystem 720

As shown in FIG. 7, processing system 722 can include one or more processors or other devices operable to control computing system 700. Such processors can include single-core processors 724, multi-core processors, which can include central processing units (CPUs), graphical processing units (GPUs), application specific integrated circuits (ASICs), digital signal processors (DSPs) or any other generalized or specialized microprocessor or integrated circuit. Various processors within processing system 722, such as processors 724 and 726, may be used independently or in combination depending on the application.

Various other configurations are may also be used, with particular elements that are depicted as being implemented in hardware may instead be implemented in software, firmware, or a combination thereof. One of ordinary skill in the art will recognize various alternatives to the specific embodiments described herein.

The various embodiments can be implemented in a wide variety of operating environments, which in some cases can include one or more user computers or computing devices which can be used to operate any of a number of applications. User or client devices can include any of a number of general-purpose personal computers, such as desktop or laptop computers running a standard operating system, as well as cellular, wireless and handheld devices running mobile software and capable of supporting a number of networking and messaging protocols. Such a system can also include a number of workstations running any of a variety of commercially available operating systems and other known applications for purposes such as development and database management. These devices can also include other electronic devices, such as dummy terminals, thin-clients, gaming systems and other devices capable of communicating via a network.

Most embodiments utilize at least one network that would be familiar to those skilled in the art for supporting communications using any of a variety of commercially available protocols, such as TCP/IP, FTP, UPnP, NFS, and CIFS. The network can be, for example, a local area network, a wide-area network, a virtual private network, the internet, an intranet, an extranet, a public switched telephone network, an infrared network, a wireless network and any combination thereof.

In embodiments utilizing a web server, the web server can run any of a variety of server or mid-tier applications, including HTTP servers, FTP servers, CGI servers, data servers, Java servers and business application servers. The server(s) may also be capable of executing programs or scripts in response requests from user devices, such as by executing one or more web applications that may be implemented as one or more scripts or programs written in any programming language, such as Java®, C, C# or C++ or any scripting language, such as Perl, Python or TCL, as well as combinations thereof. The server(s) may also include database servers, including without limitation those commercially available from Oracle®, Microsoft®, Sybase® and IBM®.

The environment can include a variety of data stores and other memory and storage media as discussed above. These can reside in a variety of locations, such as on a storage medium local to (and/or resident in) one or more of the computers or remote from any or all of the computers across the network. In a particular set of embodiments, the information may reside in a storage-area network (SAN) familiar to those skilled in the art. Similarly, any necessary files for performing the functions attributed to the computers, servers or other network devices may be stored locally and/or remotely, as appropriate. Where a system includes computerized devices, each such device can include hardware elements that may be electrically coupled via a bus, the elements including, for example, at least one central processing unit (CPU), at least one input device (e.g., a mouse, keyboard, controller, touch-sensitive display element or keypad) and at least one output device (e.g., a display device, printer or speaker). Such a system may also include one or more storage devices, such as disk drives, optical storage devices and solid-state storage devices such as random-access memory (RAM) or read-only memory (ROM), as well as removable media devices, memory cards, flash cards, etc.

Such devices can also include a computer-readable storage media reader, a communications device (e.g., a modem, a network card (wireless or wired), an infrared communication device) and working memory as described above. The computer-readable storage media reader can be connected with, or configured to receive, a computer-readable storage medium representing remote, local, fixed and/or removable storage devices as well as storage media for temporarily and/or more permanently containing, storing, transmitting and retrieving computer-readable information. The system and various devices also typically will include a number of software applications, modules, services or other elements located within at least one working memory device, including an operating system and application programs such as a client application or web browser. It should be appreciated that alternate embodiments may have numerous variations from that described above. For example, customized hardware might also be used, and/or particular elements might be implemented in hardware, software (including portable software, such as applets) or both. Further, connection to other computing devices such as network input/output devices may be employed.

Storage media and other non-transitory computer-readable media for containing code, or portions of code, can include any appropriate media known or used in the art, including storage media and communication media, such as but not limited to volatile and non-volatile, removable and non-removable media implemented in any method or technology for storage of information such as computer-readable instructions, data structures, program modules or other data, including RAM, ROM, EEPROM, flash memory or other memory technology, CD-ROM, digital versatile disk (DVD) or other optical storage, magnetic cassettes, magnetic tape, magnetic disk storage or other magnetic storage devices or any other medium which can be used to store the desired information and which can be accessed by a system device. Based on the disclosure and teachings provided herein, a person of ordinary skill in the art will appreciate other ways and/or methods to implement the various embodiments.

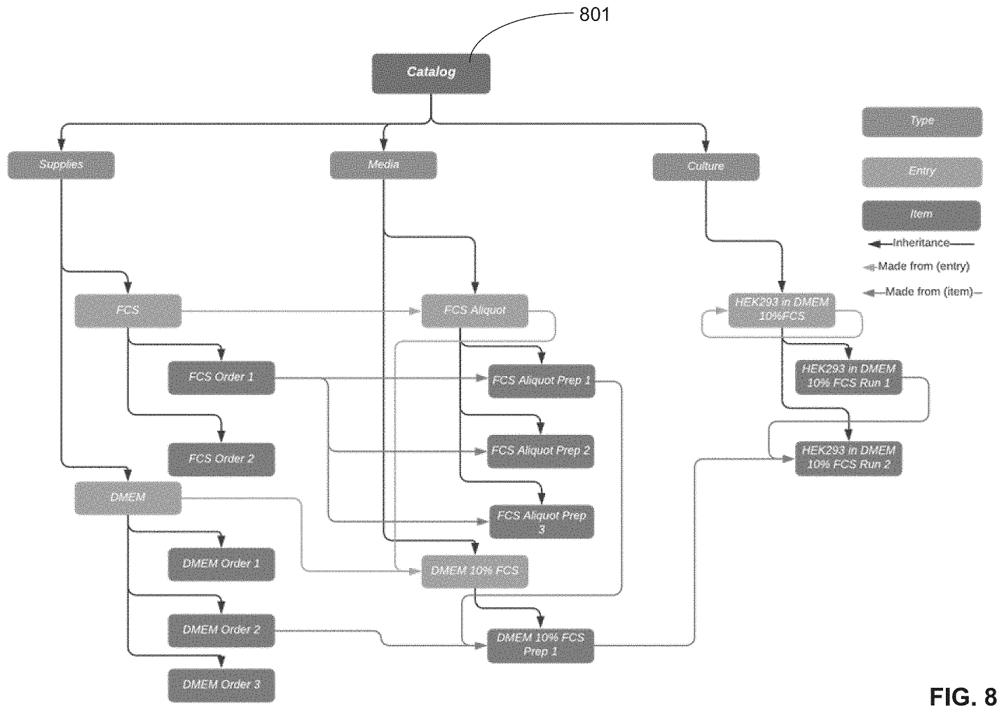

FIG. 8 illustrates, in an example embodiment, a hierarchical organization of laboratory data in a catalog structure 801. The hierarchical structure of catalog 801 as depicted includes a number of advantages:

Genericity: The catalog type/entry/item system is extremely generic. It can be applied to capture a broad range of information. In addition to its use for inventory management, it has been used to keep track of complex datasets like sequencing data, versions of new protocols, or the design of genetic constructs. It has also been successfully used to track compliance obligations like recurring training of employees, biosafety inspections, institutional biosafety committee protocols. It has also been used to keep track of general laboratory management tasks such as refilling liquid nitrogen dewars, 5S inspections, or reporting the status of hazardous waste stations.

Consistency: The catalog hierarchical organization and inheritance rules ensure that the laboratory data are consistent and maintainable.

Record lineage: Links between records of certain types make it possible to automatically create graphs to analyze the lineage of any record. It makes it possible to quickly identify what other records have contributed to a specific record. For example, it is possible to track the link between a sequencing dataset and the library preparation kit, the cell culture used to produce the data but also the media preparation used for the cell culture, and the supplies used in the preparation of the media used in the cell culture etc. Conversely, it is possible to track all the records derived from another record such as looking at all the sequencing datasets related to a particular cell culture media. The relation between records can be explored both forward and backward.

In the particular example depicted in FIG. 8, this lab purchases DMEM and FCS from vendors (supplies). The FCS is split into aliquots to limit freeze/thaw cycles. FCS Aliquots are recorded as a type of media. DMEM 10% FCS is a media made by combining DMEM and an FCS aliquot. This media is used to grow HEK293 (culture). We can see that the HEK293 in DMEM 10% FCS Run 2 is derived from DMEM 10% FCS Run 1 used as inoculum and DMEM 10% FCS Prep 1, which was prepared by combining DMEM Order 2 and FCS Aliquot 1.

This data model address some flaws of the data model used by other laboratory information management systems (LIMS) products including:

Predefined object classification: Some LIMS come with predefined classes of objects with limited flexibility to modify their data model. For example supplies and samples may be different classes of objects. Some systems may have predefined data for cell cultures, cell lines, or plasmids. Such rigid data models have led to the emergence of specialized LIMS products to manage specific object types not covered by general purpose LIMS. For example, there are LIMS for managing greenhouses or mouse colonies. One of the challenges of adopting a new LIMS is that most products require users to map their operations onto a series of objects predefined by the LIMS vendor. This can complicate the LIMS adoption and create recurring confusion as the LIMS users' and vendors' visions of a lab operation may not coincide.

Flat data model: Some LIMS products allow users to define custom object types but their data model is flat and unable to properly capture the complexity of the information manipulated by the laboratory. For example, if a single record type is used for mammalian cell lines, E. coli and yeast strains, it may be difficult to capture yeast strain genotypes in a form that is also used to capture data about mammalian cells or E. coli strains. Alternatively, if each cell type is described in a different kind of record, then there is a chance that their description may not be consistent and the rapid accumulation of record types will quickly be unmanageable.

No distinction between configuration and runtime data: Some LIMS provide a catalog of supplies and allow users to associate multiple orders with a catalog entry. However, this feature is limited to supplies and does not apply to other kinds of data. This makes it impossible to distinguish between configuration data that define the record and data collected during the record production. The consequence of this simplification is that it is virtually impossible to compare runtime data to nominal values and analyze the process stability. For example, a system that only allows users to record cell culture data without allowing them to distinguish runs of cell cultures of a certain cell type in a specific media (configuration i.e. catalog entry) will have difficulty comparing cell numbers and viability (runtime data) across multiple runs of the same culture.

Focus on inventory management: LIMS systems are designed to manage inventories of samples and supplies more than they are designed to manage complex datasets. Large and complex datasets produced by the samples saved in the LIMS are generally managed in a separate system resulting in a disconnect between samples and the data they produce. For example, it is common that large data such as sequencing data, mass spec data, or microscopy data reside on a shared drive rather than being embedded in the LIMS. Alternatively, different information management systems may be used. For example, an organization may have a system for managing sequencing data, a system for managing images, and a system for tracking samples. The link between these systems is generally weak based on naming conventions that are difficult to enforce. This makes it extremely challenging to ensure the traceability of the data.

Weak typing: LIMS systems that allow users to customize the data model of different families of records tend to offer only a limited number of simple data types based on the assumption that LIMS records are meant to be read by humans. These shortcomings make it impossible to analyze data recorded in the LIMS. For example, many LIMS do not have a good system to keep track of units. Most cannot associate tables of data to a sample. They do not have specific data types to capture the DNA or protein sequences of biological samples. Users wishing to capture this type of data have to store them in a generic text field. This makes it next to impossible to perform any kind of downstream analysis.

In accordance with catalog 801, users can develop and maintain the catalog providing data corresponding to three levels of abstraction.

Catalog types: This is the specification of the data models of different classes of objects. For example, «Supplies» and «Cell Cultures» are different classes of objects that can be described using different data.

Catalog entries: This corresponds to different objects within a catalog type. For example, “Ethanol” and “Gibson assembly kit” are examples of supplies. “HEK293 in DEMS 10%” is an example of culture.

Catalog items: These are records corresponding to the acquisition or repetitions of catalog entries. A specific bottle of ethanol corresponding to a specific order, a particular cell culture run, or media preparation would be examples of items. In general, one should expect to have multiple items associated with a single catalog entry.

Data Fields

The catalog editor allows users to specify the data associated with different catalog types as follows:

Separation of configuration and runtime data: There is a clear distinction between fields associated with a catalog entry and fields associated with the catalog items. Data associated with catalog entries define the entries; they are control parameters. Data associated with items are data specific of a particular repetition of the entry. For example, the vendor and product number and list prices are data associated with a supply catalog entry whereas the batch number, expiration date, and purchase price are data associated with a specific order (item) of a supply (entry).

Universal data fields: All catalog types have these data fields

Item Key: This is a user-defined unique prefix used to generate human-readable item unique identifiers by serializing all the items in this catalog category. For example, the type Chemicals may use CHEM as key. Ethanol being a chemical, an ethanol order will have a unique serial number of the form #CHEM00348 indicating that this is the 348th order of a chemical.

Item Name: This is the noun used to refer to items of a certain type. For example, a supply item would be called an “order” whereas a media item would be called a “preparation”.

Item Action: This allows users to specify the action necessary to obtain an item. For example, the item action for supplies may be “purchase” and the action associated with sequencing data would be “sequence”.

Supported data fields: The catalog type editor allows users to declare data fields associated with the catalog types and the corresponding items. They can specify if a field is required or optional. Several of these data fields correspond to uploads of files of a specific format. All data have a built-in data viewer apart from the generic file attachment field. The dataflow platform disclosed herein supports a broad range of advanced data types including but not limited to:
Short-text, long-text, rich text
Date and durations
URL
Currencies
Dropdown, checkboxes
Quantities organized by type of quantity (units, volume, weight) to ensure unit consistency.
Tag sets
Images and movies provided as attachments
PDF files provided as attachments supports a data viewer
Raw biological sequences: users can specify if this is a DNA or protein sequence if this is a restricted or generalized format allowing degenerate bases
Annotated biological sequences as GenBank files
Generic file attachments (does not provide data viewer)
Tables

Calculated fields allowing to calculate values derived from other fields. For example, a percentage of viable cells could be calculated from the numbers of dead and live cells.

Storage location: The type editor allows users to specify a location in the storage location hierarchy. Both catalog entries and items can have a location field. Location at the entry-level allows users to specify the container in which items should be stored. For example, they could specify that a restriction enzyme (entry) should be stored in the restriction enzyme box and a restriction enzyme order (item) would be stored at a particular position in the restriction enzyme box.

Link to other records: Catalog entries can be linked to each other. The links are directional and always indicate how records are derived from each other. For example, a cell culture type will include a link to a media and to another culture used as inoculum to specify the media and culture used to start a new culture. Links between catalog records ensure the consistency of the links at the entry and item level. For example, the catalog entry for “DMEM 10% FCS”, a growth media, would include two links to “DMEM” and “FCS”, the two supplies used to make the media. Declaring these links at the catalog entry-level creates links at the item level allowing users to specify which DMEM and FCS orders were used to prepare a batch of “DMEM 10% FCS”. When specifying a link to other records, users can specify how the quantity of the linked records should be updated when the new record is made. For example, it is possible to specify that preparing “DMEM 10% FCS” uses one 500 ml bottle of DMEM and one 50 ml FCS aliquot so that the quantities of these records can be updated when a bottle of “DMEM 10% FCS” is prepared.

Execution data: Users can specify the delay to produce the item and the labor involved. This information can be used by the automation solutions to predict the timeline of complex processes and labor needs or workload of the available labor resources.

Forms: A drag and drop form editor makes it possible to display these data fields in forms that spatially group data fields in two dimensions, provide help messages, specify what data fields should be displayed at what stage, and perform some front-end validation.

The catalog has a hierarchical structure allowing users to define increasingly specific types. Data from the parent type are inherited by the children types. Children types are defined by adding data specific to the child type on top of the data inherited from the parent type.

For example, all supplies will include data such as vendor, vendor reference, product page, and prices. Chemicals can be defined as a child type of Supplies by adding data specific to chemicals such as CAS number, Hazard categories, and a specific storage location in a chemical cabinet. Similarly, biological supplies will represent a different subtype of supplies with their own specific data such as lot number, expiration date, or storage temperature.

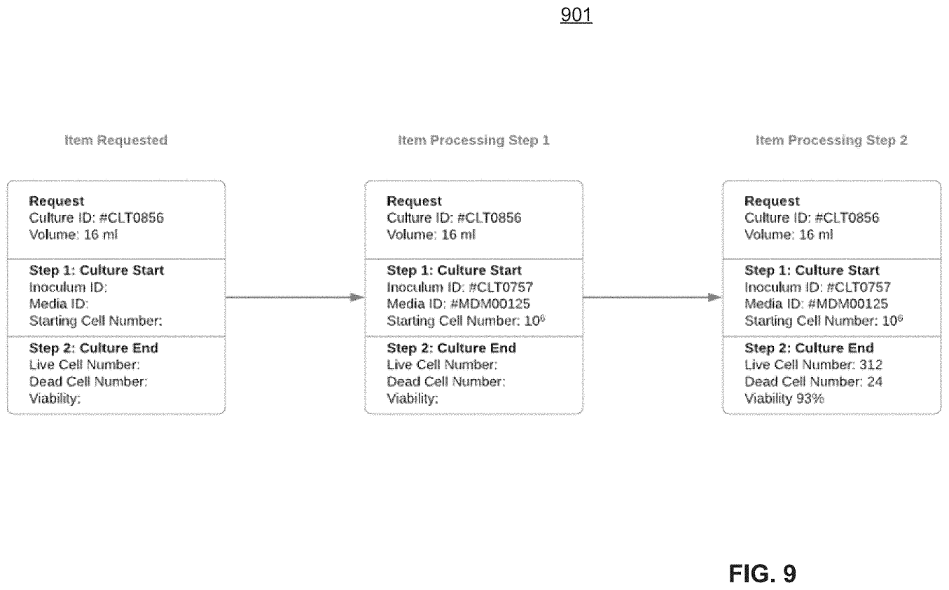

FIG. 9 illustrates, in an example embodiment, task data collected at various steps of a task completion process 901. In the embodiment depicted, various data associated with a single catalog item like a cell culture can be collected as the different steps of the process to complete the task.

Correspondence between tasks and items: A task, in embodiments depicted, corresponds to the action of “getting” an item. Users can request an item. Requesting an item creates a task. The task name is automatically generated as: “Item action”+“Catalog entry”. (i.e. Order Ethanol).

Tasks and Item Status: Tasks and Items have statuses that reflect how item requests are processed and assigned to the team.

User groups: The catalog editing tool makes it possible to specify what groups of users are allowed to place, approve, and execute item requests. For example, any lab member may be able to request the purchase of supplies in the catalog but only the lab manager has the ability to approve these requests and assign them to the procurement group. Similarly, the vector development group may have the ability to place a sequencing request, the manager of the vector group has the authority to approve these requests and assign them to the sequencing group. Members of the vector groups will only see the status of their requests whereas, members of the sequencing group will see the tasks.

Tasks steps: Users can specify the different steps of completing a task that may correspond to the different sections of a laboratory protocol. Tasks steps are defined when defining catalog types. Items-level data fields can then be associated with the different steps of the tasks to specify at which steps the data will be entered (FIG. 2). The form editor allows users to define multiple sections corresponding to the steps of the task so that only the data used to complete a step are displayed at this step and only the data captured at this step have editable fields. For example, a cell culture request can specify to use a specific cryogenic vial and specific media preparation to start the culture. The culture initiation steps can display these data and provide editable fields to capture the barcode of the media and inoculum picked up by the technician to compare them to the requests. The cell passing step would display fields to capture the number of viable and dead cells to calculate the cell number and the percentage of viable cells.

| Item Status | Meaning | Task Status | Meaning |
|---|---|---|---|
| Requested | Someone placed a request to | Backlog | The task is going to the queue |
| | obtain an instance of a catalog | | but is not assigned to anyone |
| | entry. | | yet. |
| | | To do | The task is available to pick |
| | | | up by a group of users. |
| Processing | The item request is being acted | In Progress | Someone has picked up the |
| | upon | | task and is working on it. |
| Available | The item is available for use | Done | The task has been completed |
| Canceled | The requested item has been | Canceled | The task was canceled prior |
| | canceled prior to the task | | to completion |
| | completion. The tasks may have | | |
| | failed or the request may have | | |
| | been denied | | |
| Archived | The item is no longer available | | |
| | because it's been exhausted. | | |

Computational tasks: Some items are produced using purely computational processes. For example, the design of PCR primers or the assembly of sequencing reads are the products of computational steps. Instead of being assigned to a group of technicians, these tasks are assigned to external computational resources that get input data and parameters from the LIMS and return the result of the analysis to the LIMS.

Automated Instruments: The dataflow platform, in an embodiment, is not designed to automate instrument operations. It does not allow real time control of physical processes. However, it can pass jobs and retrieve data from automated instruments that expose their services through an API as recommended by the SiLA2 standard.
Procurement tasks: Procurement tasks are a special case of computational tasks that call a procurement system or e-commerce site. Advantages include:
Associating items and tasks transforms an organization's relation with its LIMS.
Proactive LIMS: The LIMS becomes a place to assign specific jobs to members of a lab so that they can document their work as they are performing it. It tells people what to do, which helps them provide more value to their organization. It helps them do their job by giving them the information they need to complete their assignments.
Increase productivity: A task is a basic unit of productivity that supports the development of multiple dashboards. The evolution of the number of tasks completed over time provides indications of productivity trends at the organization and individual levels. A breakdown of tasks completed by members of a team may be used by managers to detect performance issues or activate a leaderboard. Users can set personal productivity goals and monitor their progress during a period of time. The distribution of tasks status over time can help diagnose resource allocation problems. For example, an ever-increasing backlog is an indication that the lab is understaffed. A growing number of tasks in processing may indicate that the staff tends to pick up tasks before completing others.
Pay Per Use Metric: Because the number of tasks is a metric clearly aligned with the value that a lab gets from the LIMS, it can be used as a metric to deploy a pay-per-use billing system. GenoFAB users pay for the software by buying task credits starting at $1 per task with volume discounts for large blocks of tasks. People can sign up to create an account at no cost to them. They can progressively adopt the LIMS by having a few individuals using it, using it for a project, one lab in a larger organization. Every account can have an unlimited number of users and unlimited data storage. All accounts can have access to all the product features at no cost to them. As they learn to recognize the product value, the number of tasks they will complete in the LIMS will increase over time creating opportunities to increase the revenue per user. Eventually, the revenue per user will be greater than the revenue per user achieved with more traditional business models but the revenue will be clearly aligned with the value users get from the product. This Pay-Per-Use experience is similar to the experience of using cloud computing platforms like AWS or payment platforms like Stripe. A low barrier to entry has gone a long way toward popularizing this new generation of computing platforms.

Limitations of existing LIMS platforms can include:

Data sinks: Historically, LIMS have been designed as data capture tools. They are designed to allow lab personnel to capture what they have done rather than to help figure out what they have to do. LIMS users tend to spend more time entering data than getting information out of their LIMS. They do their work at the bench and update the LIMS after the fact, sometimes at a much later date. This consistently leads to the LIMS being out of sync with the state of the lab. The LIMS data are often partial, inaccurate, and out of date because of the record-keeping nature of the tools.

Lack of user engagement: Laboratory personnel often have a negative image of their LIMS because they resent the clerical nature of the interaction they have with this product. They consider that their job is to work in the lab not to be data entry clerks. The value of entering data in the LIMS can be questioned because it does not benefit them directly. It may benefit someone else who will use the data. Or using the LIMS may be a necessary obligation to comply with various regulations and policies in the same way that they file their tax return to meet their taxpayer obligations.

Friction: The acquisition of a LIMS is a process plagued with a lot of friction that most potential LIMS users are not willing to overcome. Prices lack transparency. Price structures lead to restrictions in the number of users or features accessible to an organization. A LIMS license is a significant fixed recurring cost that requires a long-term commitment similar to leasing a facility. Organizations have to make this financial decision without assurance that they will get value from this investment. As a result, most organizations who could benefit from LIMS develop various sorts of avoidance strategies to avoid this investment as long as they possibly can.

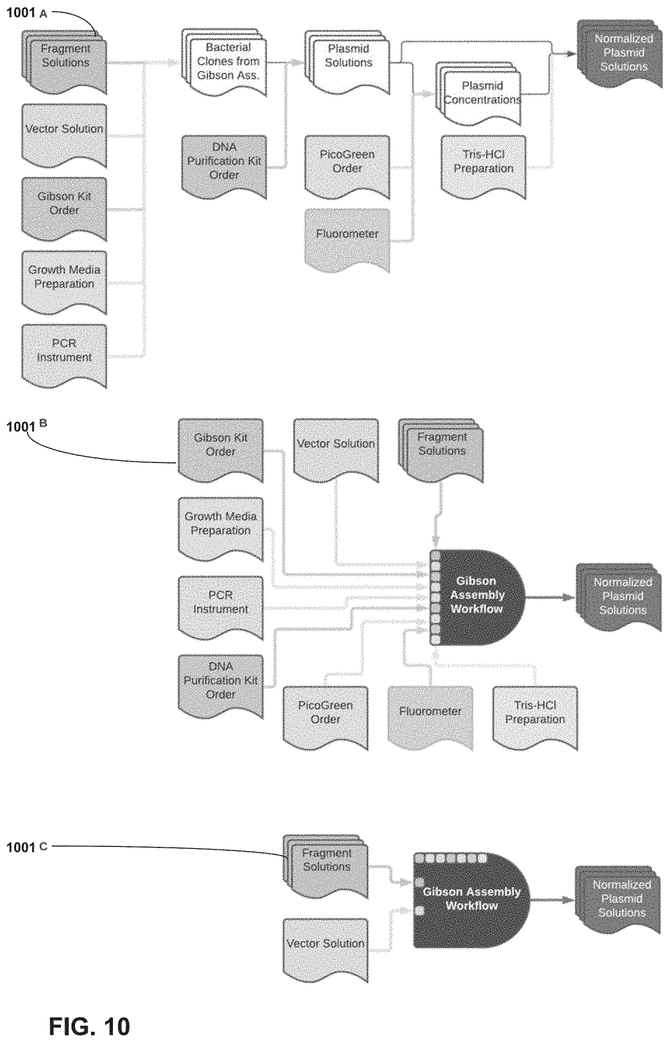

FIG. 10 illustrates, in an example, an assemble fragment step in a gene synthesis dataflow process embodiment. In the dataflow platform herein, dataflows are defined as a path on the graph defined by the links between relations between catalog records. Instead of being represented as a series of actions as in the existing approaches, the dataflow represents a sequence of data that are connected in the catalog. Since each catalog item is associated with a single task corresponding to the production of the catalog item, a data flow implicitly represents a sequence of elementary tasks.

Dataflows are defined by their interface that identifies its inputs and outputs. Workflows inputs are categorized into pushed inputs and pulled inputs. Pushed inputs are designated items used to initiate the dataflow. Pulled inputs correspond to items grabbed by the system to execute the process. Dataflow outputs can be one or more catalog items. Dataflows have no decision points.

As depicted, FIG. 10, represents the dataflow corresponding to an “assemble fragment” based on existing approaches. The first step of this workflow is the production of 12 bacterial clones by combining several DNA fragments to a plasmid solution using a Gibson kit using a PCR instrument. The resulting DNA molecule is then transformed in bacteria grown on a particular media preparation. The DNA molecules (plasmid) included in each of the bacterial clones are then extracted using a DNA extraction kit to produce plasmid solutions. The plasmid solutions are quantified using a fluorescent dye, PicoGreen and a spectrophotometer to produce a list of 12 concentrations. The concentrations are then used along with the original plasmid solutions and a buffer preparation to produce 12 new plasmid solutions having all the same concentration, in accordance with process 1001A.

It is possible to simplify this sequence of tasks by ignoring the internal steps and the corresponding data (bacterial clones, plasmid solutions, concentrations) to define a Gibson Assembly dataflow by the dataflow inputs and outputs in accordance with process 1001B. This simplification makes it possible to ignore a number of data internal to the dataflow that have no value outside of the dataflow. However, the resulting dataflow has 9 inputs.

In embodiments of the dataflow platform disclosed herein, dataflows are represented as a path connecting catalog entries. In an embodiment, a catalog entry comprises an instance of a workflow data object as referred to herein. Connectors between catalog entries represent links between catalog entries already defined in the catalog. Other data and connectors represent internal data that are not exposed by the dataflow interface. Icons representing stacks of documents represent catalog entries that include a list of simpler objects. Dataflows can have pushed inputs indicating that the dataflow is applied to a specific catalog item and pulled inputs that are retrieved from the catalog using global allocation policies defined outside of the dataflow.

Process 1001C provides a simplified representation of the dataflow that separates the inputs into two categories. Pushed inputs are represented by the connectors on the left side of the workflow icon. These connectors are used to push specific items through the workflow. Typically, a DNA assembly request will require combining a specific set of fragments using a specific vector solution. Someone requesting a DNA assembly would not require using a specific instrument or a specific assembly kit. On the other hand, the connectors on the top of the workflow icon are used to indicate that these data can be pulled from the list of available items of the corresponding catalog entries based on item allocation policies defined outside of the dataflow. For example, items the closest expiration date may be used first.

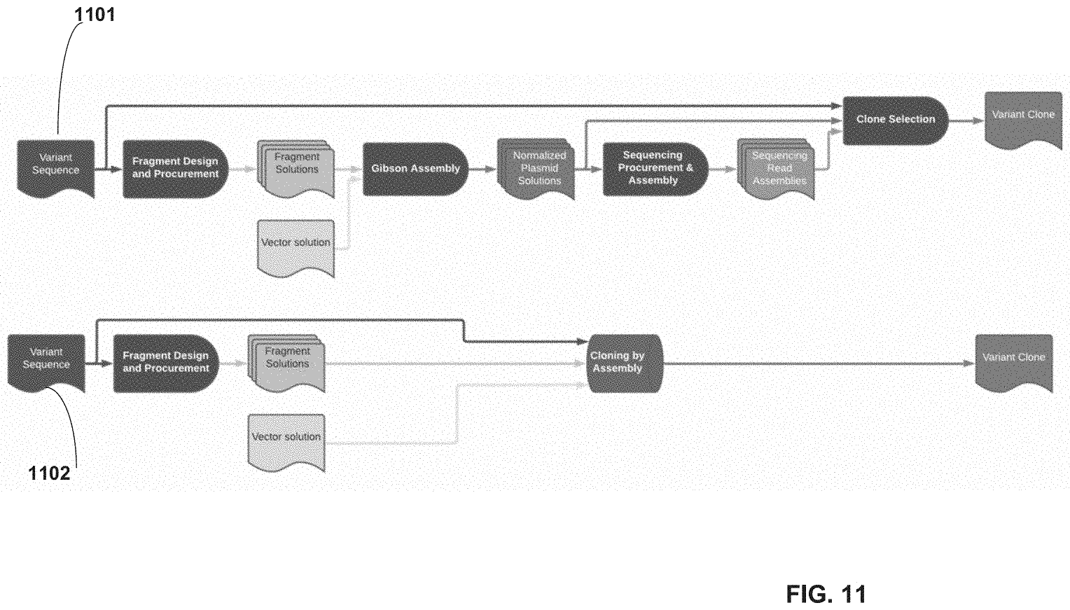

FIG. 11 illustrates an example embodiment of dataflow process hierarchy in clone matching of a design sequence. In some embodiments, the dataflow as referred to herein comprises a workflow process. Dataflow processes herein can correspond to sequences of predefined dataflows or predefined processes. This makes it possible to define increasingly complex processes hierarchically, and in this manner, abstract away the internal details of the process execution, an advantageous approach when specifying complex processes with multiple layers of abstraction.

Process hierarchy. The “synthetic fragment strategy” and the “recycling strategy” share common sequences of operations corresponding to the assembly of DNA fragments and the subsequent selection of a clone matching the design sequence. This figure shows how to go from the input sequence of a new gene variant to a clone matching this sequence using four workflows of process 1101. The dataflow platform process in this example can be defined as the sequence of the last three workflows from Gibson assembly to clone selection. The process takes the same inputs and generates the outputs as the underlying workflows. By defining this process, it becomes possible to reuse it to implement multiple assembly strategies as exemplified in process 1102.

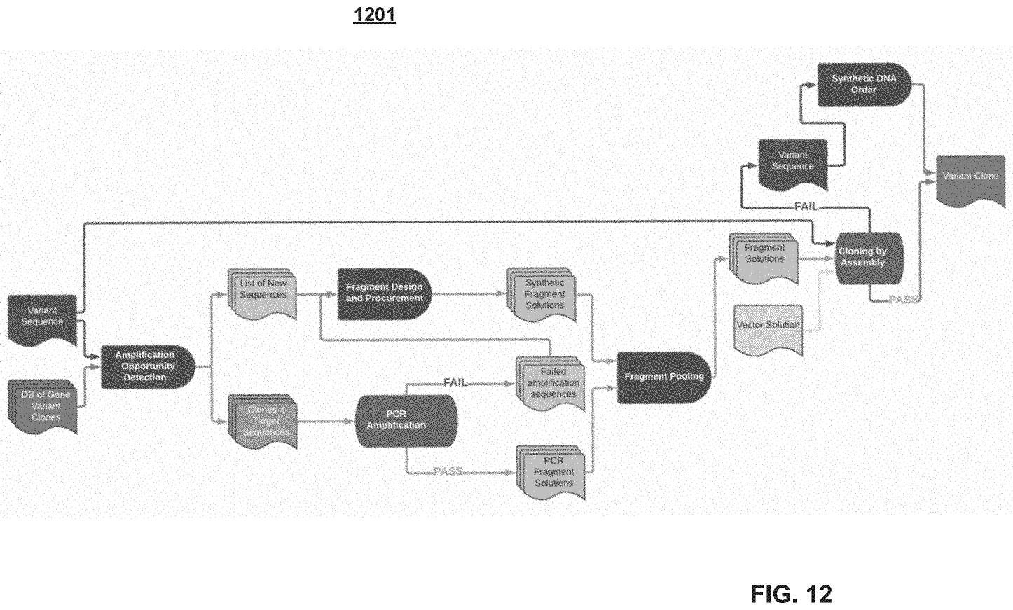

FIG. 12 illustrates, in an example embodiment, a gene synthesis laboratory dataflow process 1201. Many laboratory processes in life science have a high rate of systematic failure. Systematic failure means that the process does not fail because of a random error but because it cannot handle the inputs used to execute the process. In this situation repeating the process a second time is likely to lead to another failure. In order to properly handle these predictable failures, processes can include a success test and alternative output data depending on the result of the pass/fail test. The two outputs can be connected to different processes or workflows to specify the alternative strategy that will be taken in case of process failure.

FIG. 12 illustrates how this capability can be leveraged in the case of the gene synthesis project. Process 1201 starts by comparing the sequence of a new variant with the sequences of previously synthesized variants. A bioinformatics workflow produces a list of new fragments that will be synthesized by a vendor and a list of fragments that will be amplified from existing clones. The PCR amplification process will produce a list of DNA fragment solutions if it is successful. If it fails, it will return a list of sequences that will be ordered from a vendor since it is not possible to get these fragments from existing clones. The synthetic DNA fragments and the amplified fragments are then assembled by the Cloning by Assembly process. If the process succeeds, it returns a positive clone. However, if it fails, it returns the sequence of the new variant so that it can be ordered from a vendor. Processes can include a pass/fail test that will control the kind of data is output by the process. In this diagram the PCR amplification process can return a list of DNA fragments if it is successful. Alternatively, it can return a list of DNA sequences that failed amplification. Similarly, the Cloning by Assembly process can return a Clone if successful or a DNA sequence that failed the cloning process.

Depending on the nature of the process, the decision can be automated or manual. It can be automated by comparing the result of a QC test with a range of acceptable values. In other cases, the outcome of a process needs to be examined by an expert who will determine if the process is successful or not.

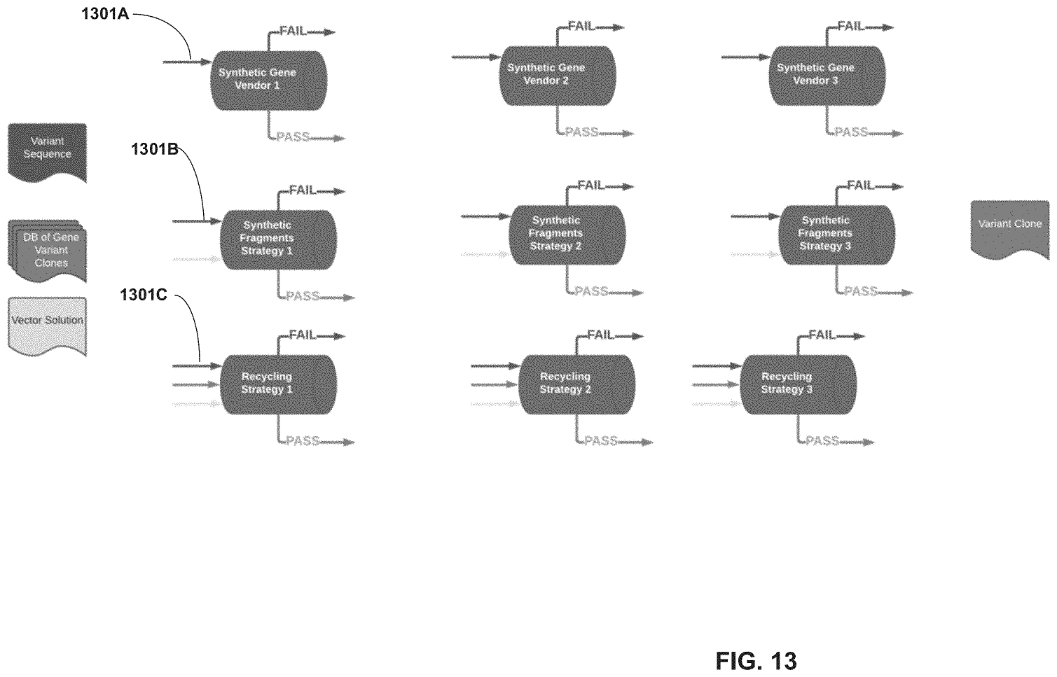

FIG. 13 illustrates, in example embodiments, dataflow based strategies 1301A, 1301B, 1301C for producing a gene variant. The problem of producing a gene variant can be solved using three dataflow strategies 1301A, 1301B, 1301C as disclosed herein. The “synthetic gene” strategy 1301A in which the gene is ordered from a vendor. The “synthetic fragment” strategy 1301B in which the gene is broken down into fragments ordered from a vendor and assembled in house. The “recycling strategy” 1301C in which the sequence of the new gene variant is first compared to the sequences of previously synthesized variants to identify opportunities to amplify existing material that may be more cost-effective than completely synthesizing the new variant.

Each of these strategies can be implemented with different services. For example, different vendors can be used to implement the “synthetic gene”, these vendors have different capabilities, different prices, and different turnaround times. Similarly, the “synthetic fragment” strategy can be implemented in different ways by using different providers of synthetic fragments, different chemistries to assemble the fragments, and different computational tools to design the fragments. The recycling strategy can also be implemented in many different ways.

FIG. 13 represents a grid of services corresponding to these three different strategies. In reality the number of services implementing each strategy is much larger than three. All these services can return a clone of the gene variant using its sequence as input. All these services can also fail in which case, they would return the variant sequence as output on the failed channel. The problem of obtaining a laboratory result like a variant clone from available resources like a variant sequence, a vector solution, and a database of available variants has many possible solutions. In accordance with the dataflow platform herein, it can be treated as a routing problem consisting in finding an optimal path through a network of services that transform data.

A lab who wishes to get a clone carrying a DNA molecule matching the gene variant sequence needs to find a path through this grid of services that connects the “variant sequence” in purple on the left to the “variant clone” in pink on the right. Without contingency plans, there are 9 possible ways of getting the gene variant using the 9 services represented in FIG. 8. Comparing these services is in itself very challenging. When considering the possibility that a service may not succeed, the optimal solution needs to include contingency plans which greatly increases the complexity of the optimization problem.

When confronted with this problem, users do not have the means to find an optimal solution. They proceed through trials and errors hoping that one of these attempts will be successful. The dataflow platform herein coordinates the laboratory processes of a large user-base has a much better perspective on the performance of individual services including cost, turnaround time, success rate, or compatibility between the input sequence and the capabilities of different services. The dataflow platform herein can leverage this information to suggest a workflow that maximizes a figure of merit set by the user. Some users may want to reduce delays, reduce cost, or maximize the use of their internal resources.

The experience would be somewhat similar to the experience of using a navigation application like Google Maps where users specify a desired destination and a starting point along with some routing parameters (avoid tolls) and Maps proposes one or a few optimal routes. The user would select one of the processes proposed by the system. As the process progresses, the process could adjust in real-time based on the outcome of some steps and the evolving conditions of the grid. This would be similar to being rerouted when making a navigation error or because traffic conditions make a route that was initially suboptimal (backroad) the best option because the optimal routing is no longer optimal (interstate at a standstill). Considering that many laboratory processes take weeks or months to complete, it is quite common that conditions change significantly during the course of a project: new service providers join the market, new technologies become available, internal resources become available, prices change.

Processes executed on the dataflow platform herein can be slow processes. Task execution is measured in hours or days and process completion in weeks, months, or even years. The dataflow platform herein advantageously automates human-driven processes. A service, whether it is a simple workflow or complex process with multiple layers of abstraction describes an acyclic graph in the hierarchical catalog disclosed herein. Each node of the graph corresponds to a single task that can be executed when all its inputs are available.

The service execution engine schedules jobs by automating the creation of catalog items and their corresponding tasks. A user submits a service request. The service request is approved by an administrator with the authority to do so. At that point, the service execution engine will create the first item request and the corresponding task. Completing a task requested by a service request sends a signal to the service execution engine that will automatically create the next task in the To Do list of the user group allowed to complete these tasks.

Advantageously, the dataflow platform herein supports two service modes of collaboration between users. In this context, collaboration refers to the ability of different labs to contribute to a complex process. Collaboration is a common challenge in the industry that takes multiple faces.

Each of these labs will have their own laboratory information system. There is considerable friction at the interface between these different labs using different information systems. It is common for data to be transferred from one lab to the other through custom file uploads or email attachments. These transfer compromise data integrity, create many opportunities for costly mistakes, and increase the workload of all parties.

It is therefore essential for the industry to create a platform allowing users to seamlessly and securely exchange data while ensuring the confidentiality of their own laboratory data. The dataflow platform herein supports two models of collaboration that achieve this goal in different ways.

In additional embodiments of the dataflow platform herein, groups can publish their services menu to specific groups of external users or to the world. Publishing a service is exposing the service interface to people outside the lab without exposing the details of the process connecting the service inputs and outputs. This interface makes it possible to pass data from one lab to another seamlessly.

For example, a large research organization may have different functional units like vector development, manufacturing, and quality control. Each unit needs to have its own catalog as they are working with different categories of objects. The people from the QC group do not want to have access to the LIMS of the vector development group. However, the groups need to collaborate by submitting service requests to each other. These services should only be accessible to a limited number of groups. A contract research organization like a sequencing facility will have a different use case. They would want their services to be accessible to a larger group of users irrespective of their affiliation with a particular organization.

The technology transfer model of collaboration aims at helping a lab reproduce a process developed by another lab. Technology transfer is typically achieved through a textual description of a process. The user manual that comes with many molecular biology kits is a good example of technology transfer. The materials and methods sections of scientific articles are another one.

This narrative approach to technology transfer is often ambiguous, difficult to understand, and lacks critical details. Finally, it can be challenging to implement the textual description of a process into a laboratory information system, which can get in the way of the successful execution of the process.

The dataflow platform transfer model allows users to import in their workspace a self-contained module that includes both a data model and a library of dataflows and processes defined over these data types. For example, a company like New England Biolabs could develop a module describing how to use their cloning and synthetic biology products, and another company may be interested in developing a sequencing library preparation module that helps users properly use their kit.

Hierarchical structure of services encourages methods validation. Laboratories operating in regulated industries are used to validating their laboratory processes using industry standards. They first develop methods, get them validated, and then use them in their operations. However, teams who are not required to validate their methods tend to take shortcuts. For example, it is common in research laboratories that people outline a complex experimental design composed of multiple steps and figure out how to execute these steps as they go. This is bad practice that increases costs and undermines reproducibility. If a team does not have the media preparation process under control, their cell culture processes will also be unstable. If their cell culture process is not reproducible, the data collected on their cell cultures will be affected by uncontrolled parameters. A better approach consists in standardizing the orders of supplies used in cell culture, then standardizing the media preparation, and then standardizing cell culture protocols. Everyone in the team should have a common understanding of these elementary methods before they can use them to collect research data. This bottoms up approach to the development of complex laboratory protocols is a sound approach to reducing costs and increasing reproducibility.

Rapid process development. Process development is an important aspect of biomanufacturing where it refers to the preliminary work to develop the process to produce a biologic drug before the process is used to actually produce the drug. Even though they may not call it that way, many life scientists spend considerable time developing processes. Many research projects involve the development of a data collection process before the process is used to collect data. A typical PhD project involves 2 years of protocol development, a year of data collection, and a year of data analysis and interpretation. The hierarchical structure of dataflow platform services disclosed herein makes it possible to rapidly develop new processes by combining previously validated building blocks and respecting data type compatibility.

Process optimization. Most laboratories need to produce data as fast as possible as cheaply as possible. The process to get the data does not matter as much as the data itself. As long as they rely on processes for which they have freedom to operate, any process that gets the data they need quickly and predictably is acceptable. Processes that are optimized by the dataflow platform in real-time based on the performance and availability of internal and external resources would give them a considerable advantage over their competitors. The competitive advantage of a research organization resides in its ability to specify what data needs to be collected to answer a scientific question rather than in its ability to collect the data themselves. Over the last 20 years, the fraction of the work that lab operators perform in their own laboratory has steadily decreased by increasingly relying on a global network of specialized service providers.

Advantage to vendors: Vendors and service providers will also benefit from the dataflow platform herein. Publishing their services will reduce their transaction costs. It would also allow them to adopt dynamic pricing strategies that better reflect market conditions and help them maximize profits. Other existing services can benefit from solutions in accordance with the dataflow platform services disclosed herein, including, but not limited to:

Laboratory Information Systems. Many LIMS offer some sort of workflow solutions. These solutions are suitable to automate simple workflows. They may be suitable for service laboratories that offer a limited number of routine services. However, they do not support hierarchical process development or selective publishing of workflows across different laboratories. They do not allow real-time process optimization using auto-routing algorithms.

Business Process Automation Systems. Software to manage and automate business processes could be considered to automate laboratory processes. There are examples of people developing LIMS in Salesforce for example. We experimented with different BPM solutions (ProcessMaker, BonitaSoft, Decisions, Taffyfy) and consistently ran into the same limitations. These tools are task-oriented. They are well-suited to businesses running a small number of processes on a large volume of cases. Developing processes in these environments is too slow and too expensive to support a process development in a life science laboratory. These tools lack the data models necessary to properly capture the dependencies between data collected at different stages of a laboratory process.

Electronic lab notebooks. Historically, scientists documented their research in paper notebooks. They meticulously documented the experiments they performed in their lab and the data they produced so that they could reproduce them. Paper notebooks suffered some numerous limitations. They were difficult to search. They were time consuming to keep, and they always lacked key information. Over the last 20 years, several products known as Electronic Laboratory Notebooks have become available. There has been a convergence between LIMS and ELN as many LIMS vendors have developed ELN solutions connected to their LIMS. Similarly, ELN developers realized that they need to offer some sort of LIMS solutions to make their product competitive on the market. ELNs are wiki-like products that for the most part, try to mimic paper notebooks. They often also offer connections with the LIMS so that notebook entries can be connected to specific samples. Despite these improvements ELNs fail to capture the evolving nature of today's scientific workflows. They are inadequate to capture the computational steps of many research projects. They are disconnected from large datasets that cannot be managed in LIMS. They flatten all structured data into a textual representation that cannot support any kind of analysis. Their linear structure makes it extremely difficult to capture the complexity of processes that may be executed on parallel tracks by different people over extended periods of time. They simply are the wrong paradigm. Process management is a better paradigm as it forces teams to specify processes in an executable form, supports the capture of data that can be analyzed to see if the process achieves the desired outcome, and allows revisions by combination of previously validated methods.

Service marketplace. The emergence of scientific service marketplaces like ScienceExchange is a sign that there is a need to streamline transactions with these vendors. The success of these projects seems limited as these marketplaces have failed to provide a framework allowing users and vendors of services to capture the information needed to quote most services.

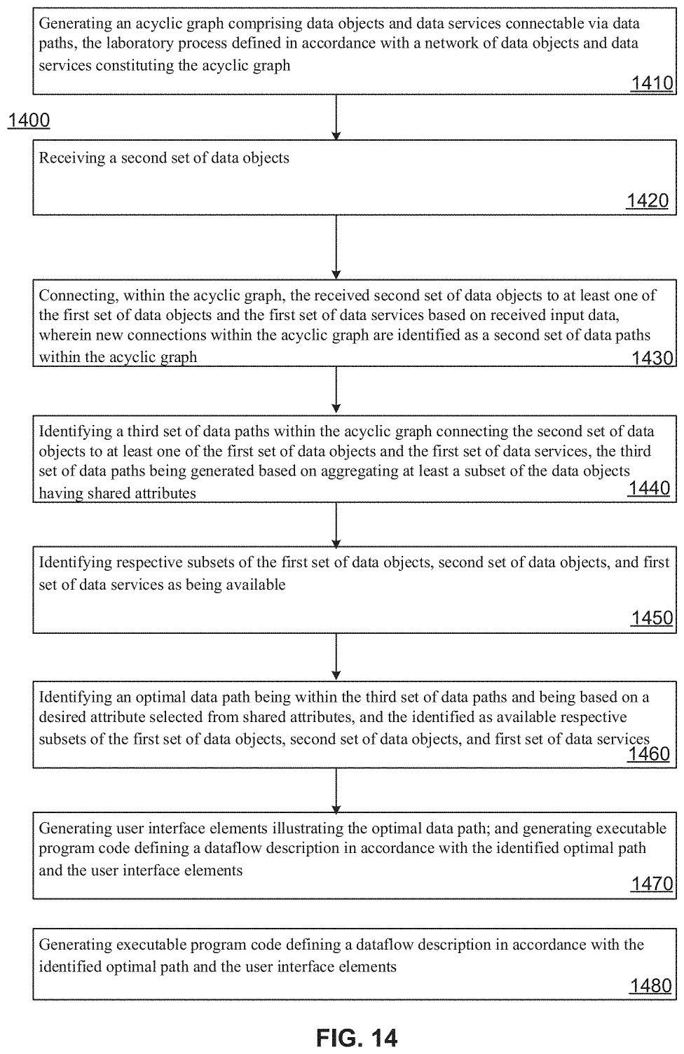

FIG. 14 illustrates an example embodiment of a dataflow process 1400. Examples of method steps described herein relate to the use of a server computing device or implementing the techniques described. Method 1400 embodiment depicted is performed by one or more processors of the server computing device. In describing and performing the embodiments of FIG. 14, the examples of FIG. 1-FIG. 12 are incorporated for purposes of illustrating suitable components or elements, including combinations thereof, for performing a step or sub-step being described.

At step 1410, generating an acyclic graph comprising a first set of data objects and first set of data services of a laboratory process, the first set of data objects and the first set of data services being connectable via a plurality of first set of data paths within the acyclic graph, the laboratory process being defined in accordance with a network of data objects and data services constituting the acyclic graph.

In embodiments, the laboratory process produces a product based at least in part on a combination of material and data inputs, the product comprising at least one of a drug, a cell line, a genetically modified organism, a mechanical device, a specialty material, and a food item.

In some aspects, the laboratory process is an analytical laboratory process comprising at least one of a quality control process, a gene synthesis process, a diagnostic process, a scientific discovery process, and an analytical process in which data produced at one step determines an outcome of a subsequent process step.

In one embodiment, the network of data objects are organized in accordance with a hierarchal catalog of records in which children data objects inherit data from parent data objects.

In one aspect, the network of data objects comprise one of a catalog type, a catalog entry, and a catalog item, wherein the catalog type defines an object data model, the catalog entry sets values of object configuration variables, and the catalog item sets values of the object execution variables.

In one variation, the network of data objects form the acyclic graph using the catalog entry as an object configuration variable of others of the network of data objects.

In some embodiments, the object configuration variable can be, for example and without limitation, a product number, a list of ingredients used to produce a laboratory sample, a set of laboratory instruments and a set software parameters.

In some aspects, the network of data services comprise data service objects in accordance with a hierarchal catalog of records in which children data services inherit data from parent data services.

In one embodiment, the network of data services comprise one of a service catalog type, a service catalog entry, and a service catalog run, wherein the service catalog type defines a service data model, the service catalog entry sets values of service configuration variables, and the service catalog run sets values of service execution variables.

In embodiments, the service execution variables can be, for example and without limitation, lot numbers, expiration dates, time stamps, the name of an operator, the measured concentration of a chemical, measured cell numbers and viability, and a microscopy image.

In one variation, the network of data services form the acyclic graph using the service catalog entries as configuration variables of others of the data services.

In another aspect, the acyclic graph defines a data service based on exposing the received input data and a received output data object, and abstracting, as the data service, a path that connects them.

At step 1420, receiving a second set of data objects.

At step 1430, connecting, within the acyclic graph, the received second set of data objects to at least one of the first set of data objects and the first set of data services based on received input data, wherein new connections within the acyclic graph are identified as a second set of data paths within the acyclic graph.

In some embodiments, the received input data is provided in response to either one of a pull operation and a push operation triggered manually from a user.

At step 1440, identifying a third set of data paths within the acyclic graph connecting the second set of data objects to at least one of the first set of data objects and the first set of data services, the third set of data paths being generated based on aggregating at least a subset of the set of data objects having at least one shared attribute.

In some aspects, the shared attribute can correspond to service execution variables as described herein. In other aspects, the shared attribute comprises at least one of a time of day, a physical location of a laboratory, a laboratory technique, a laboratory process quality metric, a laboratory protocol, an error code, and a laboratory process schedule.

At step 1450, identifying respective subsets of the first set of data objects, second set of data objects, and first set of data services as being available.

At step 1460, identifying an optimal data path, the optimal data path being within the third set of data paths and further being based on at least one desired attribute selected from the at least one shared attribute, and the identified as available respective subsets of the first set of data objects, second set of data objects, and first set of data services.

In an embodiment, the optimal data path is identified based on deploying an auto-routing algorithm across the network of data services and data objects upon interconnection with a desired output data object being specified by a user.

At step 1470, generating user interface elements illustrating the identified optimal data path; and

At step 1480, generating executable program code defining a dataflow description in accordance with the identified optimal path and the user interface elements.

In some embodiments, the method further comprises, upon receiving a desired output data object provided by a user, generating the executable program code defining the dataflow description in accordance with the identified optimal path and the user interface elements.

In one aspect, the user provides the desired output data object based on at least one of data sourced from a laboratory process instrument, a manufacturing operation, and operating a computer software application.

It is further contemplated that systems and techniques of the dataflow process as disclosed herein can also be applied beyond laboratory processes, for instance including, but not necessarily limited to, product development processes, manufacturing processes, chemical production processes, logistical processes, and inventory management processes. It is also contemplated that the dataflow process, or any portions thereof, can be implemented by way of a pay-per-use commercial model.

In some embodiments, the method steps of FIG. 14 can be performed in a processor of a server computing device, in conjunction with processor-executable instructions stored in a non-transitory, computer readable memory, the executable instructions executing the dataflow process once a complete set of process inputs become available, whether by pull or push based events.

In one specific embodiment, the present invention is comprised of a computer-implemented method for managing and optimizing a laboratory process, the method being performed in a processor of a server computing device and comprising: generating an acyclic graph comprising a first set of data objects and first set of data services of the laboratory process, the first set of data objects and the first set of data services being connectable via a plurality of first set of data paths within the acyclic graph, the laboratory process being defined in accordance with a network of data objects and data services constituting the acyclic graph; receiving a second set of data objects; connecting, within the acyclic graph, the received second set of data objects to at least one of the first set of data objects and the first set of data services based on received input data, wherein new connections within the acyclic graph are identified as a second set of data paths within the acyclic graph; identifying a third set of data paths within the acyclic graph connecting the second set of data objects to at least one of the first set of data objects and the first set of data services, the third set of data paths being generated based on aggregating at least a subset of the set of data objects having at least one shared attribute; identifying respective subsets of the first set of data objects, second set of data objects, and first set of data services as being available; identifying an optimal data path, the optimal data path being within the third set of data paths and further being based on at least one desired attribute selected from the at least one shared attribute, and the identified as available respective subsets of the first set of data objects, second set of data objects, and first set of data services; generating user interface elements illustrating the identified optimal data path; and generating executable program code defining a dataflow description in accordance with the identified optimal path and the user interface elements.

The method may be further comprised of, upon receiving a desired output data object provided by a user, generating the executable program code defining the dataflow description in accordance with the identified optimal path and the user interface elements. In one embodiment, a user may provide the desired output data object based on at least one of data sourced from a laboratory process instrument, a manufacturing operation, and operating a computer software application. In one embodiment, the network of data objects are organized in accordance with a hierarchal catalog of records in which children data objects inherit data from parent data objects. The network of data objects comprise one of a catalog type, a catalog entry, and a catalog item, wherein the catalog type defines an object data model, the catalog entry sets values of object configuration variables, and the catalog item sets values of the object execution variables. In one embodiment, the network of data objects form the acyclic graph using the catalog entry as an object configuration variable of others of the network of data objects, the object configuration variable comprising at least one of a product number, a list of ingredients used to produce a laboratory sample, a set of laboratory instruments and a set of software parameters.

In one embodiment, the network of data services comprise data service objects in accordance with a hierarchal catalog of records in which children data services inherit data from parent data services. The network of data services may comprise one of a service catalog type, a service catalog entry, and a service catalog run, wherein the service catalog type defines a service data model, the service catalog entry sets values of service configuration variables, and the service catalog run sets values of service execution variables, the service execution variables comprising at least one of a set of lot numbers, expiration dates, time stamps, name of operators or users, measured concentrations of chemicals, measured cell numbers and viability, an microscopy images.

In one embodiment, the network of data services form the acyclic graph using the service catalog entries as configuration variables of others of the data services. The acyclic graph may define a data service based on exposing the received input data and a received output data object, and abstracting, as the data service, a path that connects them.

In one embodiment, the optimal data path based on deploying an auto-routing algorithm across the network of data services and data objects upon interconnection with a desired output data object being specified by a user. In one embodiment, the laboratory process produces a product based at least in part on a combination of material and data inputs, the product comprising at least one of a drug, a cell line, a genetically modified organism, a mechanical device, a specialty material, and a food item. The laboratory process may be an analytical laboratory process comprising at least one of a quality control process, a gene synthesis process, a diagnostic process, a scientific discovery process, and an analytical process in which data produced at one step determines an outcome of a subsequent process step. The at least one shared attribute comprises at least one of a time of day, a physical location of a laboratory, a laboratory technique, a laboratory process quality metric, a laboratory protocol, an error code, and a laboratory process schedule.

In one embodiment, the received input data is provided in response to one of to a pull operation and a push operation triggered manually from a user.

In one embodiment, the invention may also be comprised of a server computing system comprising: a processor; and a memory, the memory storing instructions executable in the memory to cause operations comprising: generating an acyclic graph comprising a first set of data objects and first set of data services of a laboratory process, the first set of data objects and the first set of data services being connectable via a plurality of first set of data paths within the acyclic graph, the laboratory process being defined in accordance with a network of data objects and data services constituting the acyclic graph; receiving a second set of data objects; connecting, within the acyclic graph, the received second set of data objects to at least one of the first set of data objects and the first set of data services based on received input data, wherein new connections within the acyclic graph are identified as a second set of data paths within the acyclic graph; identifying a third set of data paths within the acyclic graph connecting the second set of data objects to at least one of the first set of data objects and the first set of data services, the third set of data paths being generated based on aggregating at least a subset of the set of data objects having at least one shared attribute; identifying respective subsets of the first set of data objects, second set of data objects, and first set of data services as being available; identifying an optimal data path, the optimal data path being within the third set of data paths and further being based on at least one desired attribute selected from the at least one shared attribute, and the identified as available respective subsets of the first set of data objects, second set of data objects, and first set of data services; generating user interface elements illustrating the identified optimal data path; and generating executable program code defining a dataflow description in accordance with the identified optimal path and the user interface elements.

The server computing system may further comprise executable instruction causing operations comprising upon receiving a desired output data object provided by a user, generating the executable program code defining the dataflow description in accordance with the identified optimal path and the user interface elements. In one embodiment of the invention, a user provides the desired output data object based on at least one of data sourced from a laboratory process instrument, a manufacturing operation, and operating a computer software application. The network of data objects may be organized in accordance with a hierarchal catalog of records in which children data objects inherit data from parent data objects. The laboratory process may produce a product based at least in part on a combination of material and data inputs, the product comprising at least one of a drug, a cell line, a genetically modified organism, a mechanical device, a specialty material, and a food item.

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

The methods, systems, and devices discussed above are described with reference to block diagrams and/or operational illustrations of methods, systems, and computer program products according to embodiments of the present disclosure. The functions/acts noted in the blocks may occur out of the order as shown in any flowchart. For example, two blocks shown in succession may in fact be executed substantially concurrent or the blocks may sometimes be executed in the reverse order, depending upon the functionality/acts involved. Additionally, or alternatively, not all of the blocks shown in any flowchart need to be performed and/or executed. For example, if a given flowchart has five blocks containing functions/acts, it may be the case that only three of the five blocks are performed and/or executed. In this example, any of the three of the five blocks may be performed and/or executed.

Specific details are given in the description to provide a thorough understanding of example configurations (including implementations). However, configurations may be practiced without these specific details. For example, well-known circuits, processes, algorithms, structures, and techniques have been shown without unnecessary detail to avoid obscuring the configurations. This description provides example configurations only, and does not limit the scope, applicability, or configurations of the claims. Rather, the above description of the configurations will provide those skilled in the art with an enabling description for implementing described techniques. Various changes may be made in the function and arrangement of elements without departing from the spirit or scope of the disclosure.

Having described several example configurations, various modifications, alternative constructions, and equivalents may be used without departing from the spirit of the disclosure. For example, the above elements may be components of a larger system, wherein other rules may take precedence over or otherwise modify the application of various implementations or techniques of the present disclosure. Also, a number of steps may be undertaken before, during, or after the above elements are considered.

Reference in the specification to “one embodiment” or to “an embodiment” means that a particular feature, structure, or characteristic described in connection with the embodiments is included in at least one example implementation or technique in accordance with the present disclosure. The appearances of the phrase “in one embodiment” in various places in the specification are not necessarily all referring to the same embodiment.

Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices. Portions of the present disclosure include processes and instructions that may be embodied in software, firmware or hardware, and when embodied in software, may be downloaded to reside on and be operated from different platforms used by a variety of operating systems.

In addition, the language used in the specification has been principally selected for readability and instructional purposes and may not have been selected to delineate or circumscribe the disclosed subject matter. Accordingly, the present disclosure is intended to be illustrative, and not limiting, of the scope of the concepts discussed herein.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the disclosure and does not pose a limitation on the scope of the disclosure unless otherwise claimed. No language in the specification should be construed as indicating any non-claimed element as essential to the practice of the disclosure.

Disjunctive language such as the phrase “at least one of X, Y, or Z,” unless specifically stated otherwise, is intended to be understood within the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Having been provided with the description and illustration of the present application, one skilled in the art may envision variations, modifications, and alternate embodiments falling within the general inventive concept discussed in this application that do not depart from the scope of the following claims.

What is claimed is:

1. A method performed in a processor of a server computing device, the method comprising:
receiving a plurality of task data objects;
generating, based on aggregating at least a subset of the plurality of task data objects, a dataflow description, ones of the at least a subset having at least one shared attribute;
generating executable program code manifesting the dataflow description in accordance with a set of nodes and links of a flow graph; and
producing an output data object based on executing, by the processor, the executable program code manifesting the dataflow description.

2. The method of claim 1 wherein ones of the task data objects are characterized in accordance with an instance of a class object specified in an object oriented programming language.

3. The method of claim 1 wherein the dataflow description relates to a laboratory process.

4. The method of claim 3 wherein the ones of the task data objects constitute a catalog tree record comprising at least one of: a cell culture, a biological sample, a genetic sequence, a protein sequence, a reagent, a scientific hypothesis, a test sequence, a clinical diagnostic, a laboratory task, and a laboratory resource device.

5. The method of claim 3 wherein the at least one shared attribute comprises at least one of a time of day, a physical location of a laboratory, a laboratory research technique, a laboratory process quality metric, a laboratory protocol, an error code, a predetermined or dynamically assigned test value or a range of values, and a laboratory process schedule.

6. The method of claim 3 wherein the laboratory process comprises a gene synthesis process, and the output data object comprises one of a positive clone of a gene variant and a deoxyribonucleic acid (DNA) sequence of a new gene variant.

7. The method of claim 3 wherein the receiving comprises receiving, responsive to a pull operation, at least one of the plurality of task data objects from a relational database.

8. The method of claim 3 wherein the receiving comprises receiving at least one of the plurality of task data objects in response to a push operation generated from a laboratory resource device in real time, the push operation being generated in accordance with at least one of a predetermined event and a dynamically triggered event.

9. The method of claim 8 wherein the executing is triggered in response to the push operation generated from the laboratory resource in real time.

10. The method of claim 1 wherein the dataflow description relates to one of: a product development process, a manufacturing process, a chemical production process, a logistical process, and an inventory management process.

11. A server computing system comprising:
a processor; and
a memory, the memory storing instructions executable in the memory to cause operations comprising:
receiving a plurality of task data objects;
generating, based on aggregating at least a subset of the plurality of task data objects, a dataflow description, ones of the at least a subset having at least one shared attribute;
generating executable program code manifesting the dataflow description in accordance with a set of nodes and links of a flow graph; and
producing an output data object based on executing, by the processor, the executable program code manifesting the dataflow description.

12. The server computing system of claim 11 wherein ones of the task data objects are characterized in accordance with an instance of a class object specified in an object oriented programming language.

13. The server computing system of claim 11 wherein the dataflow description relates to a laboratory process.

14. The server computing system of claim 13 wherein the ones of the task data objects constitute a catalog tree record comprising at least one of: a cell culture, a biological sample, a genetic sequence, a protein sequence, a reagent, a scientific hypothesis, a test sequence, a clinical diagnostic, a laboratory task, and a laboratory resource device.

15. The server computing system of claim 13 wherein the at least one shared attribute comprises at least one of a time of day, a physical location of a laboratory, a laboratory research technique, a laboratory process quality metric, a laboratory protocol, an error code, a predetermined or dynamically assigned test value or a range of values, and a laboratory process schedule.

16. The server computing system of claim 13 wherein the laboratory process comprises a gene synthesis process, and the output data object comprises one of a positive clone of a gene variant and a deoxyribonucleic acid (DNA) sequence of a new gene variant.

17. The server computing system of claim 13 wherein the receiving comprises receiving, responsive to a pull operation, at least one of the plurality of task data objects from a relational database.

18. The server computing system of claim 13 wherein the receiving comprises receiving at least one of the plurality of task data objects in response to a push operation generated from a laboratory resource device in real time, the push operation being generated in accordance with at least one of a predetermined event and a dynamically triggered event.

19. The server computing system of claim 18 wherein the executing is triggered in response to the push operation generated from the laboratory resource in real time.

20. A non-transitory computer readable memory storing instructions executable in a processor, the instructions when executed in the processor causing operations comprising:
receiving a plurality of task data objects;
generating, based on aggregating at least a subset of the plurality of task data objects, a dataflow description, ones of the at least a subset having at least one shared attribute;
generating executable program code manifesting the dataflow description in accordance with a set of nodes and links of a flow graph; and
producing an output data object based on executing, by the processor, the executable program code manifesting the dataflow description.
